(19) United States
(12) Patent Application Publication
(10) Pub. No.: US 2010/0093662 A1
(43) Pub. Date: Apr. 15, 2010

(54) NOVEL AMPHIPHILIC CYCLODEXTRIN DERIVATIVES

(75) Inventors: Jacques Defaye, Saint Ismier (FR); Alejandro Diaz Moscoso, Seville (ES); Annabelle Geze, Grenoble (FR); Pierre Vierling, Falicon (FR); Patricia Balbuena, Seville (ES); Juan M. Benito, Seville (ES); Christophe Di Giorgio, Saint-Paul (FR); Jose Garcia Fernandez, Seville (ES); Marta Gomez Garcia, Seville (ES); Carmen Ortiz Mellet, Seville (ES); Denis Wouessidjewe, Bernin (FR)

Correspondence Address: ARENT FOX LLP, 1050 CONNECTICUT AVENUE, N.W., SUITE 400, WASHINGTON, DC, 20036, US

(73) Assignee: CENTRE NATIONAL DE LA RECHERCHE SCIENTIFIQUE-CNRS-, Paris Cedex 16 (FR)

(21) Appl. No.: 12/374,211

(22) PCT Filed: Jul. 20, 2007
(86) PCT No.: PCT/FR2007/001259

§ 371 Date: Dec. 17, 2009

(30) Foreign Application Priority Data
Jul. 21, 2006 (FR) 0606719

(51) Int. Cl. A61K 31/724; C08B 37/16; C07H 21/00; C12N 5/06; A61P 43/00; A23L 1/09

(52) U.S. Cl. 514/58; 536/103; 536/23.1; 435/325; 426/661

(57) ABSTRACT

The present invention relates to cyclodextrins having the following formula (I): to nanostructures comprising them, to processes for preparing them, to the use thereof, and also to compositions comprising them.

### TECHNICAL FIELD

The present invention relates to novel cyclodextrin derivatives, and the application, thereof, in particular in the fields of pharmaceutics, cosmetics and food, and especially the transfer of nucleic acids in cells.

This invention also relates to the preparation of new cyclodextrin derivatives and the application thereof in the production of new nanosystems.

### STATE OF THE ART

Cyclodextrins, or cyclomaltooligosaccharides (CDs), are cyclic oligosaccharides known for their ability to include, in their cavity, different molecules the size of which is adapted to that of the host structure. The generally apolar nature of these associations usually results in the inclusion of hydrophobic structures. This may allow for the solubilisation in water of compounds that are barely or not soluble in water and the improvement of their stability. These properties are in particular used to improve the bioavailability of drugs.

Cyclodextrins are commercially available in three different sizes, i.e., alpha-, beta- and gamma-cyclodextrins, that comprise six, seven and eight α-D-glucopyranose residues. This availability involves a major limit concerning the size of the host molecules that may be included in their cavity. By way of example, the smallest α-cyclodextrin, may complex aromatic cycles such as benzene derivatives, whereas γ-cyclodextrin, that has a larger cavity, is able to include fused cycles such as anthracene derivatives. The largest molecules, in particular macromolecules, are in general not adapted to inclusion in cyclodextrins. In addition, the cyclodextrin: host molecule molar ratio of inclusion complexes is in general 1:1 or higher; in other words, not more than one molecule is transported per molecule of cyclodextrin.

The relatively low solubility of cyclodextrins in water, in particular commercially available cyclodextrins, and especially the most economically accessible one, beta-cyclodextrin (18 g/L, i.e. 15 mmol/l, at 25° C.), may limit the use thereof in pharmaceuticals. Moreover, since cyclodextrins have no specific ability to recognise biological receptors in the organism, these entities may thus not be used to address and vectorise active ingredients.

To remedy this fact, cyclodextrins have been chemically modified, for example primary alcohols have been substituted by monosaccharidic or oligosaccharidic groups, so as to improve their solubility in water on the one hand and, on the other hand, to incorporate cell recognition signals in their structure (international applications PCT WO 95/19994, WO 95/21870 and WO 97/33919).

However, the cyclodextrin derived from the prior art may have certain limits, in particular with respect to active ingredients likely to be transported, the load capacity of active ingredient per mass unit of cyclodextrin derivative, their ability to <<self-organise>>, their ability to <<address>> a host molecule, in particular an active ingredient, their cost, their toxicity, their ease of synthesis and/or their solubility in certain solvents, in particular in water.

Therefore, there is a need for compounds that help solve all or some of the technical problems mentioned above. In particular, in terms of load and type of compound transported as well as for the use of a simple method of preparation, with a high yield, providing a product with very high purity, at a low cost and/or that will provide a large range of compounds.

The present invention also provides new cyclodextrin derivatives that, while comprising a variety of functional groups or elements of bio-recognition or visualisation, are able to self-organise in the form of dispersible colloid systems.

### DESCRIPTION OF THE INVENTION

The present invention relates to a cyclodextrin having the following Formula (I):

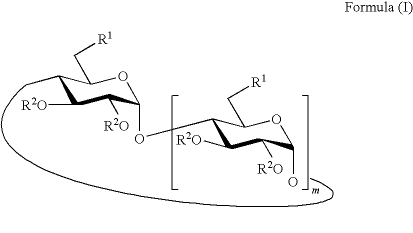

in which:
m=5, 6 or 7
the identical or different R¹ radicals represent:
   (1) an OA group where A represents a hydrogen atom, an alkyl, aryl radical, or even a protector group, such as a silyl group, in particular tert-butyldimethylsilyl or tert-butyldiphenylsilyl, in particular the OA group represents a hydroxyl group (OH);
   (2) a functional group chosen among:
a halogen atom;
an azide group (N₃);
a sulphur group of the SR³ type, in which R³ is:
   (i) an alkyl or aryl substitute or;
   (ii) an element of biorecognition such as a derivative of an amino acid, peptide, monosaccharide, oligosaccharide, an element of multiplication with several branches, the branches being able to carry identical or different glucidic groups, a visualisation or detection probe, fluorescent or radioactive, or other functional groups;
   (iii) CH₂—(CH₂)ₙ—B with n=1 to 5, B is:
NHX and X is a hydrogen atom, an alkyl or aryl group, or
NZC(=Q)NTW, where Z represents a hydrogen atom, an alkyl or aryl group, Q represents an oxygen atom or a sulphur atom and T and W, identical or different, represent a hydrogen atom, an alkyl, aryl substitute or an element of cell recognition such as an amino acid, a peptide, a monosaccharide, an oligosaccharide or even an element of multiplication with several branches bearing glucidic groups that may be identical or different, or loaded groups such as ammonium groups;
   an NHR⁴ amine group, in which R⁴ is:
   (i) a hydrogen atom;
   (ii) an alkyl or aryl substituent;
   (iii) an acyl substituent or;
   (iv) a carbamate, urea or thiourea substituent, possibly substituted by at least one group chosen among the alkyl, aryl groups, elements of biorecognition, visualisation or detection, in particular such as those mentioned above for R³
the R² radicals, identical or different, represent:
   (1) a hydrogen H, or;
   (2) an acyl, aryl or alkyl group;

with at least one of the R¹ radicals different from OH and at least one of the R² radicals different from H,

as well as their salts and isomers.

The cyclodextrins represented above by Formula (I) may relate to both per(C-6)fonctionnalised and mono(C-6)fonctionalised amphiphilic cyclodextrin derivative. In the first case, all of the R¹ groups are identical, whereas in the second case, one of the R¹ differs from the others.

The cyclodextrins according to the invention thus have an amphiphilic nature that may allow them to have a solubility of interest in different solvents, in particular in water, and/or allow them to self-organise to form systems creating colloid suspensions.

In addition, these cyclodextrins may be associated with, or form complexes with, host molecules in a host molecule/cyclodextrin ratio ranging from 1/2 to 1,000/1, in particular from 1/1 to 100/1, especially from 1.1/1 to 20/1, or even 1.5/1 to 10/1.

<<Protective group>> according to the present invention, refers to a group allowing to avoid that the function on which it is found react with the reagents used in the reactions that the compound on which it is found undergoes. In particular, this type of group is described in the works <<Protective groups in organic synthesis>>, Wiley, by T. W. Green and P. G. M. Wuts, 3^{e} ed, 1999, or <<Protective groups>>, Georg Thieme Verlag by P. Kocienski, 3^{e} ed, 2003.

<<Alkyl>> according to the present invention, refers to a carbonated, linear, branched or cyclic, saturated or unsaturated radical, in particular comprising 1 or 2 to 12 carbon atoms.

<<Aryl>> according to the present invention, refers to a radical comprising at least one aromatic cycle, possibly substituted, in particular by an alkyl. Said aryl may comprise 6 to 20 carbon atoms.

The R², R³ and/or R⁴ radicals may be chosen among the benzyl, phenyl, allyl, methyl, ethyl, propyl, butyl, pentyl, hexyl groups or superior homologues comprising up to 12 carbon atoms, linear, branched or cyclic, saturated or unsaturated, these groups may comprise other neutral or loaded functional groups.

<<Functional group>> according to the present invention, refers to a mono- or polyatomic unit with a characteristic reactivity or even a group of atoms with a specific valence and characterising a function.

The R² and/or R⁴ radicals may be chosen among the acetyl, propionyl, butyroyl, pentanoyl, hexanoyl groups or homologues comprising up to 22 carbon atoms, linear, branched or cyclic, saturated or unsaturated, these groups may carry other neutral or loaded functional groups.

<<Acyl>> according to the present invention, refers to a carbon radical comprising at least one carbonyl function, of type R^{a}—C(=0)-, in which R^{a} in particular represents an aryl or alkyl radical.

<<Bio-recognition element>> according to the present invention, refers to a molecular structure complementary to a biological receptor, able to specifically associate with the latter, in particular by non-covalent bonds, and in particular lead to a specific response, for example:
an induction or inhibition of a transduction signal induced after association with the receptor,
an induction and control of the biosynthesis of an enzyme,
an inhibition of the activity of an enzyme by fixation on its active site,
an induction of an immune response following a bacterial disease, a viral infection, and/or
an inhibition of an inflammatory process by blockage of the active site of a selectine.

<<Element of multiplication with several branches>>, according to the invention, in particular refers to a branched carbon chain comprising either a quaternary tetrasubstituted carbon atom such as the derivatives of tris(2-aminomethyl)methylamine (TRIS) and pentaerythritol, or a trisubstituted nitrogen atom such as tris(2-aminoethyl)amine (TREN). These elements of multiplication may even be incorporated in combination with a second branch element comprising, in particular, a tertiary nitrogen atom such at the derivatives of tris(2-aminoethyl)amine (TREN).

<<Visualisation or detection probe>>, according to the present invention, refers to a molecular structure allowing for the detection of a system by a physiochemical technique, such as fluorescence or radioactivity. Among the fluorescent probes, the derivatives of fluorescein, dansyl (5-(dimethylamino)-1-naphtalenesulfonyl) or coumarin can be mentioned. Among the radioactive probes, products labelled with a radioactive isotope can be mentioned.

According to one method, the aforementioned elements of cell recognition and/or the elements of biorecognition are bound to the rest of the molecule directly or by means of a spacer arm, for example an alkyl spacer arm in C₁ to C₁₀ or heteroalkyl in C₁ to C₁₀, possibly substituted. For example, the spacer arm may be —CH₂CH₂—NHC(═S)NHCH₂CH₂SCH₂—, as illustrated in examples 25 and 26.

According to one method, the amphiphilic cyclodextrin complies with Formula (I) in which all of the R¹ radicals are identical and represent halogen atoms, i.e. fluorine, chlorine, bromine and iodine and in particular chosen among iodine and bromine.

In particular, the cyclodextrin complies with Formula (I) in which at least one RI group, or even all R¹ groups, represent the radioactive isotope of iodine with an atomic mass of 129. In particular, this radioactive group may allow visualisation of the nanoparticle systems in which it is integrated. This may be especially useful to study the transport and biodistribution of active ingredients in vivo.

In particular, a cyclodextrin according to the invention complies with Formula (III):

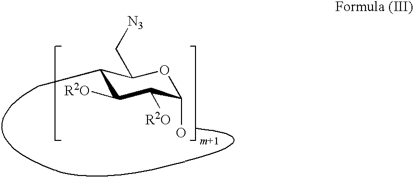

where m and R² have the meaning indicated above.

The presence of the azide group, in these derivatives, may be of interest in particular to promote the self-organisation of amphiphilic cyclodextrins in stable colloid systems of the type of nanocapsule or nanosphere.

When R¹ groups, or even all R¹ groups, represent halogen atoms and the latter are shifted by sulphur nucleophiles, it is then possible to obtain compounds complying with Formula (I) in which R¹ represents SR³, R³ having the meaning indicated above for Formula (I).

According to a specific procedure, the present invention relates to a compound complying with Formula (IV):

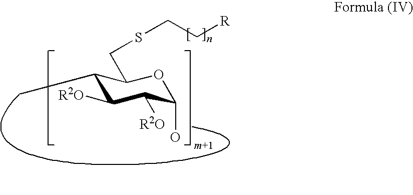

in which m and R² have the meaning indicated above,

n=1, 2, 3, 4 or 5 and R represents an amine function such as:

(i) an NHY amine group, Y representing a hydrogen atom or an alkyl, acyl or carbamate substituent; or
(ii) a quaternary —NY₃ ammonium group, Y representing an alkyl substituent.

In particular, a compound according to the invention complies with Formula (IV) in which n=2, and R represents the tert-butoxycarbonylamino (NHBoc) group or NH₂, the resulting amine group may be protonated.

In these derivatives, the presence of ω-aminoalcanethiol spacer group and in particular cysteaminyl, may help give rise to polycationic amphiphilic derivatives.

The presence of a ω-aminoalcanethiol spacer group, and in particular cysteaminyl, may also help increase the reactivity of the amine groups, in particular in the case of compounds complying with Formula (IV) with R═—NHY, Y representing a hydrogen atom, an alkyl or aryl.

Moreover, it should be noted that this spacer group may be introduced easily by using commercially available cysteamine or a homolog ω-aminoalcanethiol as reagent. In particular, this avoids the stage of reduction required when the amine groups are prepared from an azide precursor as is the case in the examples described in document WO 97/33919.

This prefunctionalised spacer group may help associate cyclodextrin with a hydrophilic unit and cell recognition such as a glucidic derivative, or even an amino acid or a peptide, by urea, thiourea, amide and thioether bonds that are very stable and give rise to well defined structures.

The thiourea bond is created in a final stage, thus helping couple cyclodextrin with a great many substituents, in particular substituents comprising an element of multiplication with several branches bearing different glucidic units, and/or loaded groups, and/or a fluorescent or radioactive probe for visualisation or detection. These amphiphilic thioureidocysteaminyl-cyclodextrins are original products that show a remarkable affinity with respect to complementary lectins.

The cyclodextrins according to the invention, in particular ureido- and thioureidocysteaminyl-cyclodextrins, may be represented by the following Formula (V):

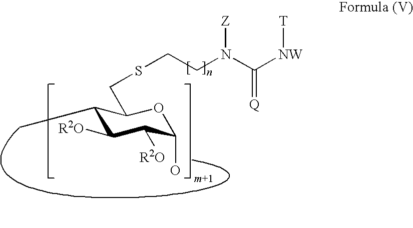

in which m and R² have the meaning indicated above, and
n represents a whole number chosen among 1, 2, 3, 4 or 5,
Z represents a hydrogen atom, an alkyl or aryl group,
Q represents an oxygen atom or a sulphur atom and
T and W, identical or different, represent a hydrogen atom, an alkyl, aryl substituent or an element of cell recognition such as an amino acid, a peptide, a monosaccharide, an oligosaccharide or even an element of multiplication with several branches bearing glucidic groups that may be identical or different, in particular bearing substituents, or loaded groups such as ammonium groups, in particular protonated —NHY groups or —NY₃ groups, Y representing a hydrogen atom, an alkyl or aryl substituent.

In particular, when T and/or W represent an alkyl substituent, this substituent is an alkyl with 1 to 12 linear, branched or cyclic carbon atoms.

When T and/or W represent an aryl group, the latter may in particular be chosen among phenyl, benzyl, naphtyl or derivatives of these groups bearing other substituents on the aromatic ring.

When T and/or W represent a substituent derived from monosaccharides, the latter may also be a derivative of glucose, mannose and galactose, in α or β form.

The group derived from the monosaccharide may be substituted, in particular one or several hydroxyl groups from the monosaccharide may be replaced by alcoxy groups with 1 to 16 carbon atoms, acyloxy groups, for example the acetoxy group, amine and amide groups.

When T and/or W represent oligosaccharide derivatives, the oligosaccharide derivative groups may be maltosyl, maltotriosyl, lactosyl, or even tri- or tetrasaccharide groups, in particular Lewis X or sialyl Lewis X cell affinity markers, or even oligosaccharide derivatives of heparin. These oligosaccharide derivative groups may also be substituted by alcoxy, acyloxy groups, amino, sulphate or phosphate groups.

When T and/or W represent a group comprising an element of branched multiplication, this element may be a derivative group of tris(2-hydroxymethyl)methylamine (TRIS), pentaerythritol or tris(2-aminoethyl)amine (TREN).

The branches of these branched multiplication elements may comprise derivatives of identical or different mono- or oligosaccharides. By way of example, it is possible to mention derivative groups of mono- ou oligosaccharides indicated in the previous paragraph that may also comprise oxygen or amine substituents. These glucidic groups may be bound to a multiplication element by an oxygen, sulphur or amine bond.

According to a specific procedure, at least one of the branches comprises a probe, in particular a fluorescent or radioactive probe, in particular allowing for the visualisation or detection of the system.

A specific compound according to the invention complies with Formula (V) in which n=2, Q represents a sulphur atom, Z and T represent a hydrogen atom and W represents the methyl group.

Other specific compounds according to the invention comply with Formula (V) where m=6, n=2, Q represents a sulphur atom, Z and T represent a hydrogen atom and W respectively represents, (i) the 2-hydroxyethyl group, (ii) the 2-(tert-butoxycarbonylamino)ethyl group, (iii) the 2-aminoethyl group, the amine group may be protonated or (iv) the 2-(α-D-mannopyranosyloxy)ethyl group.

Other specific compounds according to the invention comply with Formula (V) where m=6, n=2, Q represents a sulphur atom, Z and T represent a hydrogen atom and W represents the branch element 2-[2-azidoethyl-2′-(tert-butoxycarbonylamino)ethyl]aminoethyl or 2,2-bis[2-(tert-butoxycarbonylamino)ethyl]aminoethyl.

Another specific compound according to the invention complies with Formula (V) where m=6, n=2, Q represents a sulphur atom, Z represents a hydrogen atom, T and W are identical and represent the 2-(tert-butoxycarbonylamino)ethyl group.

Another specific compound according to the invention complies with Formula (V) where m=6, n=2, Q represents a sulphur atom, Z represents a hydrogen atom, T represents the 2-azidoethyl group and W represents the 2-(tert-butoxycarbonylamino)ethyl group.

The invention also refers to derivatives complying with Formula (V) where at least one of the two substituents T and W represent a substituent bearing other functional groups, in particular loaded groups such as ammonium groups, in particular protonated —NHY or —NY₃ groups, Y representing a hydrogen atom, an alkyl or aryl substituent.

In particular, the cyclodextrins comply with Formula (V) where all of the R² radicals represent a hexanoyl group.

In a specific procedure, the compounds complying with Formula (IV) where Y represents a hydrogen atom or an alkyl or aryl substituent as well as the derivatives complying with Formula (V) where at least one of the substituents T and W carry an NHY group, Y having the same meaning as mentioned above, may be isolated in the form of ammonium salts or a free base.

In the case of salts, the counter-ion may be a monovalent anion, in particular a halide such as chloride, bromide or iodide. The free base like the salt may be used as precursors in the preparation of amphiphilic thioureidocysteaminyl-cyclodextrins.

In a preferred procedure, the compounds comply with Formulae (I) to (V), where all the R² radicals represent the hexanoyl group and/or the tetradecanoyl (myristoyl) group and m=6.

According to one method, the compound according to the invention complies with the following Formula (VI):

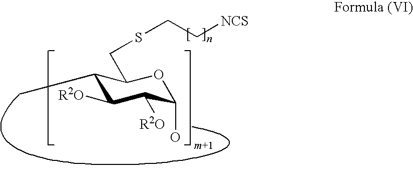

in which
n=1, 2, 3, 4 or 5, and m and R² have the meaning indicated above,
the NCS group represents the isothiocyanate group.

Specific compounds according to the invention comply with Formula (VI) where n=2, m=6 and R² respectively represents the hexanoyl group and tetradecanoyl (myristoyle) group.

According to another procedure, the compound according to the invention complies with the following Formula (VII):

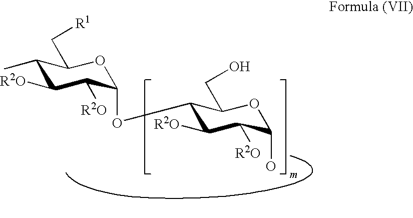

in which m, R¹ and R² have the meaning indicated above.

The cyclodextrin corresponding to Formula (VII) presents hydroxyl groups on the carbon in position 6 of each of the monomers, except for one of the monomers where the carbon in C6 bears an R¹ substituent different from the hydroxyl group.

Specific compounds according to the invention comply with Formula (VII) where the R¹ group respectively represents the 2-(tert-butoxycarbonylamino)ethylthio group, the 2-aminoethylthio group, the 2-[N′-(2-α-D-mannopyranosyloxyethyl)thioureido]ethylthio group and the 2-(N′-(2-(cyclomaltoheptaose-6^{I},-desoxy-6^{I}-yl)ethylthio)thioureido)ethylthio group.

Specific compounds according to the invention correspond to Formula (VII) where m=6 and all of the R² groups represent a hexanoyl group.

In particular, the invention relates to cyclodextrins according to the invention likely to develop in particular non-covalent interactions with, at least one host molecule, in particular thus forming an inclusion complex.

The present invention also relates to the preparation of the compounds (cyclodextrins) described above.

The method of synthesis is very flexible as regards the type of R¹ and R² groups. In particular, it may help optimise the amphiphilic characteristics, the ability to self-organise in an aqueous medium, the properties of the resulting colloid systems and/or the ability to complex, among others, small molecules or macromolecules.

The method for the preparation of cyclodextrin according to the invention comprises the following stages consisting in:

(i) introducing at least one R² group on at least one of the carbons bearing the primary hydroxyl or protecting at least one of the primary hydroxyls in the initial compound, in particular a cyclodextrin;
(ii) introducing at least one R² group on at least one secondary hydroxyl carried by the carbon in position 3 of the monomers forming a cyclodextrin; and
(iii) recovering at least one cyclodextrin according to the invention, in particular a compound from Formula (I), obtained.

According to a first variant, the procedure may include a stage prior the introduction of R² consisting in protecting at least one primary hydroxyl, or even all of the primary hydroxyls.

The protector group initially introduced from a commercially available cyclodextrin may be a silylether group, in particular tert-butyldimethylsilylether, in particular in the case of per(C-6) functionalised derivatives and when the R² groups represent an alkyl subsituent.

The primary hydroxyls may in particular be protected by reaction with the chloride from tert-butyldimethylsilyl and the imidazole in an aprotic polar solvent, preferably N,N-dimethylformamide.

The selectively silyl derivatives on the primary OH may then be alkylated by reaction with an alkyl halide, in particular in an aprotic polar solvent such as N,N-dimethylformamide, in the presence of a base, in particular sodium hydride.

Subsequently, the silyl groups may be hydrolysed, in particular by the action of an aqueous acid, preferably acetic acid or trifluoroacetic acid, or even by treatment with a fluorhydric acid salt, preferably tetra-n-butylammonium fluoride.

The resultant non-protected derivatives, presenting free primary OH functions and, bearing alkyl chains on the secondary OH, may be transformed into other derivatives bearing different functional groups on the primary side according to the methods of transformation of the primary hydroxyls, in particular halogenation. For this, it is possible to follow the procedures indicated above for the fonctionalisation of the commercially available cyclodextrins on the primary side.

According to a second variant, the method may include a stage prior to the introduction of R² consisting in substituting at least one primary hydroxyl, or even all of the primary hydroxyls, by an R¹ group.

In particular, the method may include a stage of halogenation of the cyclodextrins on at least one carbon bearing a primary hydroxyl, or even on all the carbons bearing a primary hydroxyl.

In particular, the method according to the invention comprises a stage of substitution of primary OH by halogenated group, preferably iodine or bromine, followed by acylation of at least one secondary hydroxyl, or even all secondary hydroxyls, to provide a compound of Formula (I) where R² represents at least one acyl group.

The cyclodextrin derivatives persubstitued in primary alcohol position by the halogenated groups may, in particular, be prepared using commercially available cyclodextrin in a single stage and with good yields by reaction with different selective halogenation reagents. For this, it is possible to use the methods described by J. Defaye and al. in the documents Supramol. Chem., 2000, 12, pp. 221-224, Polish J. Chem. 1999, 73, pp. 967-971, Tetrahedron Lett. 1997, 38, pp. 7365-7368, Carbohydr. Res. 1992, 228, pp. 307-314, and Angew. Chem., Int. Ed. Engl. 1991, 30, pp. 78-80.

The per(C-6)halogenated cyclodextrins may undergo an acylation reaction of secondary OH in a second stage, in particular to obtain compounds of Formula (I) where at least one R² group represents an acyl.

It is possible to use the procedure described by P. Zhang and al. in Tetrahedron Lett. 1991, 32, 2769-2770. However, this procedure may require long reaction times and high temperatures, producing modest or even low yields in the case of these halogenated derivatives.

According to another method, per(C-6)halogenated cyclodextrins may be acylated by reaction with an acid anhydride in an aprotic polar solvent, in particular N,N-dimethylformamide, in the presence of a base, in particular N,N-dimethylaminopyridine. In these conditions, the reaction may be complete in 45 min. at ambient temperature, with yields in pure product of about 70%.

In a specific procedure, the preparation of cyclodextrins complying with Formula (I) in which all of the R¹ groups represent a halogen atom, with Formula (III) and Formula (IV) where R represents NHY, Y represents an acyl or carbamate group and the R² radical represents an acyl group, may be carried out according to the method consisting in having a cyclodextrin derivative, selectively halogenated, azidated or fonctionnalised with NHY in primary alcohol position react with an acid anhydride, in particular in N,N-dimethylformamide, in the presence of a base, preferably N,N-dimethylaminopyridine.

The per(C-6)halogenated amphiphilic cyclodextrins complying with Formula (I) in which all R¹ are identical may be used as starting products in the preparation of derivatives incorporating other functional groups on the primary side.

The halogenated groups, that are good starting groups, may be shifted by nitrogenous or sulfurated nucleophilic groups, such as the azide anion, amines or thiols. It is possible to use the procedures described by J. Defaye and al. in the documents Carbohydr. Res. 1995, 268, 57-61, J. Chem. Soc., Perkin Trans., 2, 1995, pp. 1479-1487 and WO2004087768.

The compounds in Formula (III) may be prepared from a precursor complying with Formula (I) where the R¹ groups are all halogen atoms by reaction with an azide anion.

Alternatively, the cyclodextrins in Formula (III) may be obtained by acylation of the corresponding per(6-azido-6-desoxy)cyclodextrin. It is possible to use the procedures described by P. Zhang and al. in Tetrahedron Lett. 1991, 32, 2769-2770 or the new method using N,N-dimethylformamide indicated above for the acylation of per(C-6)halogenated cyclodextrins.

Thus, in a specific method, the procedure to prepare the cyclodextrin in Formula (III) consists in having a halogenated derivative of the cyclodextrin in Formula (I) where R¹ is a halogen atom react with an azide anion in N,N-dimethylformamide.

The cyclodextrins in the invention complying with Formula (III) may also be used as starting products in the preparation of other neutral or loaded derivatives due to the reactivity of the azide group.

Therefore, an addition reaction of 1,3-dipolar catalysed by the copper (II) cation with an alkyne may be used to bind a variety of substituents by means of a 1,2,3-triazol cycle. The procedure described by Santoyo-Gonzàlez and al. in Org. Lett. 2003, 5, 1951-1954 may be used.

Moreover, the reduction of azide groups may lead to the corresponding per(C-6)amine. This reduction may be obtained by a variety of procedures such as hydrogenation in the presence of a heterogenous palladium, platinum or nickel catalyst, reaction with a phosphine, preferably triphenyl or tributylphosphine, or reaction with propanedithiol. The procedures described by J. Defaye and al. in Carbohydr. Res. 1995, 268, 57-61 or by L. Jicsinszky and al. in Comprehensive Supramolecular Chemistry, Vol. 3 (Eds J. Szejtli and T. Osa), Pergamon, Oxford, 1996, pp. 57-198 may be used.

The compounds in Formula (IV) may be prepared from a compound in Formula (I) where the R¹ radicals are identical and represent halogen atoms by reaction with a sulfurated nucleophile, in particular of formula HSCH₂(CH₂)ₙR.

The procedure described in document WO2004087768 may be used.

Alternatively, a neutral compound of Formula (IV) (R═NHY, where Y=acyl or carbamate) may be obtained by acylation of the corresponding derivative where R² represent H, obtained as described in the aformentioned document, by acylation. The method described by P. Zhang and al. in Tetrahedron Lett. 1991, 32, 2769-2770 or the new procedure using N,N-dimethylformamide indicated above may be used for the acylation of per(C-6)halogenated cyclodextrins. This method does not directly provide the loaded derivatives. However, this type of derivative may be obtained from the corresponding carbamates, preferably the tert-butoxycarbonyl cyclodextrins (Formula IV where R=NHBoc) by acid hydrolysis of the carbamate function.

In a specific procedure, the method to prepare cyclodextrin in Formula (IV), consists in reacting a cyclodextrin derivative in Formula (I) in which the R¹ radicals are identical and represent halogen atoms with cysteamine, a ω-aminothiol, or one of their derivatives, in particular in N,N-dimethylformamide, in the presence of a base, such as triethylamine or cesium carbonate.

In another specific procedure, the method to prepare cyclodextrins complying with Formula (IV) where R represents a primary amine group (NH₂) consists of the hydrolysis of the carbamate group in a precursor of Formula (IV) where R represents an NHBoc group.

The compounds of Formula (V) (ureido- or thioureidocysteaminyl-cyclodextrins) may be prepared by a method comprising stages consisting in having a compound of Formula (IV) where R represents NHY, with Y representing a hydrogen atom or an alkyl or aryl substituent, react with an isocyanate or isothiocyanate of formula W—NCQ (Q represents an oxygen atom or a sulphur atom, respectively) where W has the meaning provided above.

This reaction may be carried out in an organic solvent such as pyridine or even in a mixture of water with a miscible organic solvent such as acetone. This provides a compound of Formula (V) (ureido- or thioureidocysteaminyl-cyclodextrin) where T represents a hydrogen atom.

When Y represents H in Formula (IV), the thioureas obtained are N,N′-disubstituted, while when Y represents a alkyl substituent, such as methyl, ethyl, propyl or butyl, the thioureas obtained are N,N,N′-trisubstituted.

The isothiocyanate compound W—NCS may be prepared in different ways:
when W is a group derived from a monosaccharide or an oligosaccharide, by reaction of thiophosgene on an aminodesoxyglycose or a glycoside comprising an amine group in aglycone. The procedures described by J. M. Garcia Fernandez and C. Ortiz Mellet in Adv. Carbohydr. Chem. Biochem. 1999, 55, pp. 35-135 may be used.
when W comprises a branched multiplication element derived from tris(2-hydroxymethyl)methylamine (TRIS), the corresponding isothiocyanate may be prepared by reaction of thiophosgene on the amine derivative bearing glucidic substituents on the primary alcohol positions, as described in document Chem. Commun., 2000, pp 1489-1490. The trivalent amine glycodendron precursor may be obtained by glycosidation of a TRIS derivative with the suitably protected amine function in carbobenzoxy derivative form, as described by P. R. Ashton and al. in J. Org. Chem. 1998, 63, pp 3429-3437.
when W comprises a branched multiplication element derived from tris(2-aminoethyl)amine (TREN), the corresponding isothiocyanate may be prepared by reaction of thiophosgene on a selectively protected derivative on two primary amine groups, for example by the Boc group. The procedure described by Benito and al. in J. Am. Chem. Soc. 2004, 126, 10355-10363 may be used. It is also possible to use a derivative bearing two different protector groups, such as the Boc group and the trifluoroacetyl group. These types of branch elements may be prepared by selective protection using commercially available TREN or by reaction of bis(2-aminoethyl)amine, selectively protected on primary amine groups by the groups mentioned, with 2-azidoethyl-p-toluenesulfonate, followed by the reduction of the azide group and then reaction with thiophosgene.
when W comprises a branched element of multiplication derived from pentaerythritol, the glycodendrons suitably functionalised with an isothiocyanate group may be prepared with commercially available pentaerythritol by a sequence of reactions involving:
   (a) selective triallylation by treatment with allyl bromide, providing only one free hydroxyl group;
   (b) the radicular addition of a 1-thiosugar with the double bond of the allyl groups.

This reaction may be obtained, either by activation by ultraviolet light, or in the presence of an initiator of free radicals such as azobis(isobutyronitrile) or p-nitroperbenzoic acid. By way of example, the reactive conditions described by D. A. Fulton and J. F. Stoddart in Org. Lett. 2000, 2, pp 1113-1116 or by X.-B. Meng and al. in Carbohydr. Res. 2002, 337, pp 977-981 may be adapted.

This reaction may allow for the sequential addition of different glucidic branches.

It is thus possible to obtain homogenous as well as heterogenous glycodendrons (polyconjuguated, that comprise several identical or different glucidic units, bound covalently to a branched nucleus, the branched nucleus moreover bearing a functional group enabling it to be grafted on another platform) in which the glucidic substituents comply with the structures indicated above. This procedure has been described by Gômez-Garcia and al. in J. Am. Chem. Soc. 2005, 127, 7970-7971.

This approach also enables the incorporation of a substituent other than a glucidic derivative in the structure, in particular a fluorescent probe such as a fluorescein derivative.

(c) the transformation of the primary alcohol group remaining in the isothiocyanate group. This transformation may, for example, occur through the transformation of the hydroxyl group into a good starting group such as p-toluenesulfonate or trifluoromethanesulfonate, followed by nucleophile shift by azide anion and isothiocyanation of the resulting azide by reaction with triphenylphosphine and carbon disulphide. For this transformation, the procedure described in document Chem. Commun., 2000, pp 1489-1490 may be used.

The compounds complying with Formula (V) (thioureiocysteaminyl-cyclodextrins) where Q represents a sulphur atom and Z represents a hydrogen atom may also be prepared using a precursor complying with Formula (VI), corresponding to Formula (I) where m and R² have the meaning indicated above and the R¹ represent the —SCH₂(CH₂)NCS, group with n=1, 2, 3, 4 or 5.

These compounds in Formula (VI) may be obtained from the corresponding amine from Formula (IV), where m and R² have the meaning indicated above with n=1, 2, 3, 4 or 5 and where R represents NHY, Y represents a hydrogen atom, by reaction with an agent of isothiocyanation, preferably thiophosgene.

The coupling of a product from Formula (VI) with a primary or secondary amine of general formula WNHT, when T and W have the meaning indicated above, provides a compound of Formula (V) (thioureidocysteaminyl-amphiphilic cyclodextrin).

In a specific procedure, the method to prepare compounds of Formula (V) where Q represents a sulphur atom and Z represents a hydrogen atom consists in having a precursor complying with Formula (VI) react with an amine WNHT, W and T having the meaning indicated above.

The compounds corresponding to Formula (VII) where one of the substituents R¹ differs from hydroxyl and the others represent OH, may be prepared according to the procedure consisting in:

(i) selectively introducing a functional group on one of the primary positions of the cyclodextrin;
(ii) protecting the other primary hydroxyls with a protector group, in particular in silylether form;
(iii) then introducing the substituents on the primary hydroxyls; and
(iv) hydrolysing the protector groups, if need be.

Based on 6^{I}-p-tolylsulfonyl cyclodextrins, a specific means of production of compounds complying with Formula (VII) (preparation described in WO 99/61483), consists in:

(i) shifting the p-toluenesulfonate group by a nucleophile, in particular halide, azide, sulphur derivative or amine derivative;
(ii) protecting the remaining primary OH, in particular in the form of silylether groups, in particular tert-butyldimethylsilylether;
(iii) acylating or alkylating secondary OH, and;
(iv) hydrolysing or fluorolysing silylether groups to regenerate the corresponding hydroxyls.

The compounds of Formula (VII) (cysteaminyl-amphiphilic cyclodextrin derivatives) where m, and R² have the meaning indicated above and R¹ represents SCH₂(CH₂)ₙR, n and R having the meaning indicated above for the compounds of Formula (IV), may be obtained using a monosubstituted cyclodextrin derivative in primary alcohol position by a halogenated group, in particular the 6^{I}-bromodesoxy or 6^{I}-desoxyiodo-cyclodextrins. The reaction with a cysteamine derivative protected on the amine group, preferably by the Boc group, or, in general, with a derivative of an N-protected, possibly N-alkylated ω-aminoalcanethiol, according to the method described above for the preparation of persubstituted derivatives in primary alcohol position, provides monocysteaminyl-cyclodextrins. The above mentioned sequence of reactions to obtain derivatives of Formula (VII) provides the amphiphilic derivatives of the invention.

The cyclodextrin derivatives monosubstituted in primary alcohol position by halogenated groups, used as starting products in this method, may be prepared using the 6^{I}-0-p-tolylsulfonylated derivative of cyclodextrin in one stage and with a good yield by nuclophilic shift of the tosylate group by a halide anion in N,N-dimethylformamide.

In general, the procedures for the preparation of per(C-6)substituted amphiphilic cyclodextrins are also applicable to the preparation of derivatives monofonctionalised in primary position, i.e., substituted at the level of a carbon bearing a primary hydroxyl.

The processes for the preparation of cyclodextrins according to the invention and described above have the advantage of helping provide desired derivatives in the form of pure and homogenous products in a reduced number of stages and with high yields. In addition, these procedures provide both neutral and polycharged, in particular polycationic derivatives that may bear elements of biorecognition or visualisation.

The novel cyclodextrin derivatives according to the invention, while bearing a variety of functional groups or elements of bio-recognition or visualisation, may be able to self-organise, in particular in the form of dispersible colloid systems.

In addition, the possibility of incorporating different groups into these novel derivatives on the side of the primary alcohols is of interest, in particular to modulate the interaction properties of the outer surface of nanoparticle (nanocapsule or nanosphere) colloid systems that they provide.

Thus, according to another of its aspects, the invention refers to nanostructures comprising at least one cyclodextrin of Formula (I) according to the invention, possibly comprising at least one host molecule.

In particular, these nanostructures may incorporate, comprise, be associated with or form a complex, with at least one host molecule.

The nanostructures may in particular come in the form of nanospheres, nanocapsules and or nanoparticles.

The nanostructures, in particular the nanospheres and/or nanocapsules may in addition comprise at least one host molecule.

The host molecule may be any active ingredient, in particular a pharmacologically active molecule, for example drugs or nucleic acids.

The host molecule may be a molecule chosen in the group comprising nutriments, trace elements, vitamins, flavours.

The host molecule may even be a molecule that may be used in cosmetology, in particular the molecules indicated in the “International Cosmetic Ingredient Dictionary and Handbook” by John A. WENNINGER and G. N. McEWEN.

The host molecule may also be a nucleic acid, in particular selected from the group comprising DNA, RNA, modified nucleic acids such as the ribonucleotides or the desoxyribonucleotides presenting a sugar group or a modified carbon group, or even synthetic analogs of nucleotides.

The nanocapsules may enclose or contain an organic phase, in particular Miglyol 812 (trade mark).

The nanoparticles may comprise at least one nucleic acid, in particular selected in the group comprising DNA (linear or plasmic), RNA (and in particular the interfering RNA -ARNi- or even <<silencing>> -RNAsi-, micro-ARN), modified nucleic acids, such as the ribonucleotides or the desoxyribonucleotides presenting a sugar group or a modified carbon group, or even synthetic analogs of nucleotides.

In particular, the cyclodextrins complying with Formula (I) where R¹ represents a halogen atom, present an excellent self-organisation in nanocapsule or nanosphere stable colloid systems.

Moreover, the neutral compounds complying with Formulae (I) to (VII) have been found to be especially interesting as regards their self-organisation ability, in particular in nanocapsules or nanospheres, in stable colloid systems.

<<White nanosphere>> or <<white nanocapsule>> according to the present invention refers to nanospheres or nanocapsules not loaded in a host molecule, and in particular in an active ingredient.

The nanostructures, in particular the nanospheres, in particular white and/or homogenous nanospheres, i.e. in which the cyclodextrins are all of the same type, in particular all presenting the same formula, may be prepared according to the method comprising the stages consisting in:

(i) adding a water-miscible organic solvent, containing at least one compound from Formula (I) to an aqueous solution, the volume of water varying by one to two times the volume of organic solvent, while shaking;
(ii) then after nanoprecipitation, i.e. the formation of nanospheres, eliminating the organic solvent.

The aqueous suspension obtained that may have an opalescent to milky appearance (according to the products used), may then be concentrated to the final volume desired.

The procedure for the preparation of nanospheres presented above may be applied to the preparation of heterogenous nanospheres. For this, stage (i) of the preparation presented above consists of the introduction in an aqueous solution, of at least two water-miscible organic solutions, containing different amphiphilic cyclodextrin derivatives, in particular different chemical formulae, in variable proportion while shaking. The volume of aqueous solution may range from one to two times the volume of organic solvent.

This method of preparation of heterogenous nanospheres is used to incorporate loaded amphiphilic cyclodextrins in the nanosphere even when these derivatives used alone do not form nanospheres.

The nanostructures, and in particular the nanocapsules, in particular the white nanocapsules, i.e. the nanocapsules that do not incorporate active ingredient, may be prepared by a method comprising stages consisting in:

(i) preparing an acetonic phase containing a small fraction of triglycerides, preferably a proportion of acetone: triglycerides ranging from 1,000:1 to 10:1, at least one amphiphilic cyclodextrin, at least one lipophilic non ionic surfactant, and a hydrophilic phase containing distilled water and at least one hydrophilic non ionic surfactant;
(ii) introducing the organic phase in the hydrophilic phase with magnetic stirring;
(iii) after formation of the nanocapsules (nanoprecipitation), elimination of the organic solvent, in particular under reduced pressure at +35° C.

In particular, in stage (i), at least two preparations of cyclodextrins, in variable proportion, containing cyclodextrins of Formula (I) of different chemical formulae are added.

The aqueous suspension presenting a milky appearance may then be concentrated until the final volume desired is obtained.

According to another aspect, the invention refers to a method for the preparation of nanostructures, in particular nanospheres and/or nanocapsules, comprising, containing or bearing at least one host molecule as defined above, the method comprising the following stages:

(i) the introduction of at least one organic phase containing a water-miscible solvent, such as acetone, a cyclodextrin derivative, or alternatively, several preparations respectively comprising cyclodextrins complying with Formula (I) of different chemical formulae in variable proportion, and the active ingredient, in an aqueous phase, in particular containing distilled water, possibly with a surfactant, in particular a hydrophilic non ionic surfactant, while stirring,
(ii) then after nanoprecipitation, i.e. the formation of nanocapsules or nanospheres, the elimination of the organic solvent.

According to another aspect, the present invention refers in particular to cosmetic, dietary and/or pharmaceutical compositions comprising at least one cyclodextrin and/or one nanostructure according to the invention and a host molecule, in particular a pharmacologically active molecule.

In particular, the invention refers to a pharmaceutical composition containing per unit dose of 50 mg to 500 mg of cyclodextrin and/or nanostructures according to the invention and a pharmacologically active host molecule in a molar proportion of cyclodextrin derivative/host molecule that may range from 50:1 to 1:500, in particular from 25:1 to 1:10, in particular from 20:1 to 1:1, or even 10:1 to 1.5:1.

A major advantage of the present invention, resides in the fact that the self-organisation in systems of colloid nanoparticles (nanospheres or nanocapsules) helps significantly increase the load capacity in active ingredient, well beyond a 1:1 molar proportion. The active ingredient may be located in the cavity of the cyclodextrin or at the surface or in the matrix of nanospheres or even in the lipophilic core of the nanocapsules.

For classic, non nanoparticulate cyclodextrins, it should be noted that there may be a major limit in the size of the molecules likely to be encapsulated, a limit determined by the size of the hydrophobic cavity. The limit is about 900 Dalton (D) of molar mass. The maximum load capacity is 1:1 in molar base. We go way beyond this with the nanoparticle systems. For example, plasmids of 4000 kD have been encapsulated.

The invention also refers to cyclodextrin derivatives able to form stable nanostructures of several tens or hundreds of nanometres by desolvation in the presence of a non solvent (nanospheres), possibly in the presence of a lipophilic phase (nanocapsules). These colloid suspensions take up large quantities of host molecule, in a molar proportion of cyclodextrin derivative/host molecule that may range from 50:1 to 1:500, in particular from 25:1 to 1:10, in particular from 20:1 to 1:1, or even from 10:1 to 1.5:1, and in particular help (i) modify, improve and control the pharmacokinetic properties of pharmacologically active ingredients, (ii) transport active compounds, and (iii) facilitate or improve the intracellular transfer of active compounds.

The cyclodextrins and/or nanostructures according to the invention may be used for the complexation of nucleic acids and/or for the introduction of nucleic acids in cells.

The cyclodextrins and/or nanostructures according to the invention may also be used to facilitate and/or improve the intracellular transfer of a host molecule and/or to control the release of a host molecule.

The cyclodextrins and/or nanostructures according to the invention may also be used for the introduction of nucleic acids in cells, in particular eukaryotes.

According to a specific procedure, the nanoparticles according to the invention also comprise a nucleic acid.

Nucleic acid refers to the DNA (linear or plasmic), the RNA (and in particular the interferent RNA -ARNi or even <<silencing>> -RNAsi-, micro-RNA) or modified nucleic acids, in particular ribonucleotides or desoxyribonucleotides presenting a sugar group or a modified carbon group. The nucleic acids may be used in single strand, double strand or partially double strand form.

The nucleic acids may also comprise or contain synthetic analogs of nucleotides, in particular ribonucleotides presenting a modified sugar group or carbon group. For example, the synthetic analogs of ribonucleotides presenting a modified sugar group present a 2′-OH group replaced by a group selected among a hydrogen atom, a halogen, an OR, R, SH, SR, NH₂, NHR, NR₂ or CN group, where R is an alkyl, alkenyl or alcynyl group of 1 to 6 carbons and the halogen is fluorine, chlorine, bromine or iodine. The ribonucleotides presenting a modified carbon group may have their phosphoester group bound to the adjacent ribonucleotide i.e. replaced by a modified group such as a phosphothioate group. The ribonucleotides may be ribonucleotides presenting a purine or modified pyrimidine core. Examples of such modified cores, in particular include the uridines or the cytidines modified in position 5, such as 5-(2-amino)propyl uridine and 5-bromo uridine, the adenosines and guanosines modified in position 8, such as 8-bromo guanosine, the denitrogenous nucleotides, such as 7-deazaadenosine, N- and 0-alkyl nucleotides, such as N6-methyl adenosine.

The inventors in particular noted as regards the cyclodextrins of Formula (V) that when the urea or thiourea group carries positively loaded groups, synergy effects in the complexation of nucleic acids may occur, the cyclodextrins then being optimised for the introduction (transfection) of genetic material in cells.

The polycationic amphiphilic cyclodextrin derivatives are especially effective in complexing nucleic acids. These derivatives self-organise around a nucleic acid in aqueous medium, giving rise to the formation of nanoparticles that enclose the nucleic acid and that are able to cross the cell membrane, as demonstrated in the examples, to release the nucleic acid that may then express its biological action inside the cell.

One specific procedure in the invention refers to an in vitro process of transfer of host molecules in cells, in particular eukaryote cells consisting in:

(i) putting the nanostructure/host molecule complex and/or the cyclodextrin/other host molecule complex in contact with cells;
(ii) leaving the cells in contact with the nanostructure/host molecule complex and/or cyclodextrin/host molecule complex for a time T preferably between 4 and 72 hours;
(iii) removing the culture medium and washing the cells.

The time of contact may depend on the cyclodextrins used to form the nanostructures, in particular the presence of elements of bio-recognition that may specifically bond with a membrane receptor and thus facilitate the penetration of the cyclodextrin/host molecule complex and/or nanostructure/host molecule complex in the cell.

The optimum time of contact may easily be determined by a person skilled in the art by carrying out experiments at different times, for example 4, 8, 12, 24, 36, 48, 60 and 72 hours.

The incorporation of elements of biorecognition, for example, the ligands complementary to a receptor, allow the derivatives incorporating them to be very effectively recognised by the specific membrane receptors. In particular, an element of glucidic recognition will be specifically recognised by a lectin as a function of the glucidic substituents incorporated, in this way allowing for the vectorisation of small molecules or macromolecules at the level of target cells.

The invention also refers to a pharmaceutical composition comprising either (i) an amphiphilic cyclodextrin derivative of Formula (I) or (ii), a colloid system according to the invention prepared from one or several amphiphilic cyclodextrins of Formula (I), (iii) or a complex of an amphiphilic cyclodextrin derivative of Formula (I) or (iv), a colloid system prepared from one or several amphiphilic cyclodextrins of Formula (I) and a pharmacologically active molecule, preferably with a pharmacologically acceptable carrier.

The invention also refers to a pharmaceutical composition comprising (i) an amphiphilic cyclodextrin derivative of Formula (I) according to the invention and/or a nanostructure according to the invention and (ii) a nucleic acid, in particular a DNA fragment, preferably with a biocompatible polymer such as polyethyleneglycol.

The composition may also comprise an addressing element, such as a glucid or peptide derivative or even a derivative of a ligand specifically bonding with a cell receptor, in particular folic acid, or even transferrine. This addressing element may integrate in the amphiphilic cyclodextrin-DNA nanoparticle or may anchor on this system by inclusion of a hydrophobic unit carried by the element of vectorisation, for example a substituent derivative of adamantane, in the cavity of the cyclodextrin.

The pharmaceutical compositions of the invention that may be administered by oral or parenteral route, may in particular be in the form of solutions, powders, suspensions.

Other characteristics and advantages of the invention appear clearer when reading the following non limiting examples provided by way of illustration.

### Example 1

### Preparation of heptakis(6-desoxy-2,3-di-0-hexanoyl-6-iodo) cyclomaltoheptaose (compound no, 1)

This compound complies with Formula (I) where all R¹ are identical, with m=6, R¹═I and where R² represents the hexanoyl group.

A solution of heptakis(6-deoxy-6-iodo)cyclomaltohepaose (0.5 g, 0.26 mmol) in dried DMF (20 mL), in argon, at 0° C., is added to N,N-dimethylaminopyridine (DMAP, 1.35 g, 11.0 mmol, 3 eq). The hexanoic anhydride (3.4 mL, 14.7 mmol, 4.0 eq) is then added drop by drop and the reactive mixture is shaken for 45 nm (minute) at ambient temperature. The reaction is completed by the addition of MeOH (25 mL). This mixture is again shaken for 1 hr (hour), then poured into glacial water (50 mL) and extracted with CH₂Cl₂ (4×50 mL). The organic phase is washed in turn with diluted sulphuric acid (2 N; 2×50 mL) and a saturated solution of NaHCO₃ (4×50 mL), then dried (Na₂SO₄), concentrentrated and purified by silica gel column chromatography with a mixture of EtOAc-petroleum ether ->1:10 as eluent. This thus provides compound no. 1 (0.58 g, 68%) with the following characteristics:
R_{f}=0.40 (EtOAc-petroleum ether 1:5)
[α]_{D}=+63.8 (c 1.0, CHCl₃)

### Example 2

### Preparation of heptakis(6-bromo-6-desoxy-2,3-di-O-hexanoyl)cyclomaltoheptaose (compound no. 2)

This compound complies with Formula (I) in which all R¹ are identical, with m=6, R¹═Br and in which R² represents the hexanoyl group.

Compound no. 2 is obtained by acylation of heptakis(6-bromo-6-desoxy)cyclomaltoheptaose (0.61 g, 0.39 mmol) in dried DMF (29 mL) with DMAP (2.0 g, 16.3 mmol, 3 eq) and the hexanoic anhydride (5.0 mL, 21.7 mmol, 4.0 eq), according to the method indicated above for the preparation of compound no. 1. The purification of the reactive mixture by chromatography on silica gel column with an EtOAc-petroleum ether mixture of 1:10→1:8 as eluent provides compound no. 2 (715 mg, 63%) with the following characteristics:
R_{f}=0.60 (EtOAc-petroleum ether 1:8)
[α]_{D}=+73.3 (c 0.8, CHCl₃)

### Example 3

### Preparation of heptakis(6-azido-6-desoxy-2,3-di-O-hexanoyl)cyclomaltoheptaose (compound no. 3).

This compound complies with Formula (III) with m=6 and in which R² represents the hexanoyl group.

A solution of heptakis(6-azido-6-desoxy)cyclomaltoheptaose (0.2 g, 0.15 mmol) in dried DMF (20 mL), in argon, at 0° C., is added to N,N-dimethylaminopyridine (DMAP; 783 mg, 6.4 mmol, 3 eq). The hexanoic anhydride (2.0 mL, 8.5 mmol, 4.0 eq) is then added one drop at a time and the reactive mixture is shaken for 16 hrs at ambient temperature, then concentrated under reduced pressure and diluted with CH₂Cl₂ (20 mL). The resulting solution is washed with diluted sulphuric acid (2 N; 2×10 mL) and concentrated. The residue is taken up by MeOH (15 mL) and a saturated aqueous solution of NaHCO₃ (50 mL) is added. The mixture is shaken at ambient temperature for 1 h, then extracted with CH₂Cl₂ (3×30 mL), the organic phase is dried (Na₂SO₄), concentrated, and the residue purified by silica gel column chromatography with an EtOAc-petroleum ether mixture 1:15→1:9 as eluent. Compound no. 3 is thus obtained (286 g, 71%) with the following characteristics:
R_{f}=0.42 (EtOAc-petroleum ether 1:9)
[α]_{D}=+115.0 (c 1.0, CH₂Cl₂)
IR: vₘₐₓ, 2117 cm⁻¹

### Example 4

### Preparation of heptakis(6-desoxy-6-iodo-2,3-di-O-myristoyl)cyclomaltoheptaose (compound no. 4).

This compound complies with Formula I in which all R¹ are identical, with m=6, R¹═I and in which R² represents the tetradecanoyl (myristoyl) group.

A solution of heptakis(6-desoxy-6-iodo)cyclomaltoheptaose (100 mg, 53 μmol) in dried DMF, in argon (5 mL), at 0° C., is added to N,N-dimethylaminopyridine (DMAP, 269 mg, 2.2 mmol, 3 eq). The tetradecanoyl anhydride (1.29 g, 2.94 mmol, 4.0 eq) is then added and the resulting suspension is shaken for 16 hr at ambient temperature and then filtered. The resulting solid is washed by distilled water and then MeOH, then heated to reflux in a CH₂Cl₂-MeOH mixture (5:95, 50 mL) for 1 hr, decanted and purified by silica gel column chromatography with a petroleum ether -> mixture of EtOAc-petroleum ether 1:12->1:10 as eluent. Compound no. 4 is thus obtained (0.58 g, 68%) with the following characteristics:
R_{f}=0.49 (EtOAc-petroleum ether 1:8)
[α]_{D}=+49.2 (c 1.0, CH₂Cl₂)

### Example 5

### Preparation of heptakis(6-bromo-6-desoxy-2,3-di-O-myristoyl)cyclomaltoheptaose (compound no. 5).

This compound complies with Formula I in which all R¹ are identical, with m=6, R¹=Br and in which R² represents the tetradecanoyl (myristoyl) group.

Compound no. 5 is obtained by acylation of heptakis(6-bromo-6-desoxy)cyclomaltoheptaose (100 mg, 63.5 μmol) in dried DMF (5 mL), with DMAP (326 mg, 2.67 mmol, 3 eq) and the tetradecanoyl anhydride (1.56 g, 3.55 mmol, 4.0 eq), according to the method described above for the preparation of compound no. 4. The purification of the reactive mixture by silica gel column chromatography with a petroleum ether -> EtOAc-petroleum ether mixture gradient 1:12 as eluent provides compound no. 5 (193 mg, 67%) with the following characteristics:
R_{f}=0.31 (1:8 EtOAc-petroleum ether)
[α]_{D}=+49.3 (c 1.0, CH₂Cl₂)

### Example 6

### Preparation of heptakis(6-azido-6-desoxy-2,3-di-0-myristoyl)cyclomaltoheptaose (compound no. 6).

This compound complies with Formula (III) with m=6 and in which R² represents the tetradecanoyl (myristoyl) group.

A solution of heptakis(6-azido-6-desoxy)cyclomaltoheptaose (100 mg, 76.3 μmol) in dried DMF (7 mL), in argon, at 0° C., is added to N,N-dimethylaminopyridine (DMAP; 392 mg, 3.2 mmol, 3 eq). The tetradecanoyl anhydride (1.87 g, 4.3 mmol, 4.0 eq) is then added and the resulting suspension is shaken for 16 hrs at ambient temperature and then filtered. The resulting solid is washed by distilled water, then heated to reflux in a CH₂Cl₂-MeOH mixture (5:95, 25 mL) for 1 hr, and filtered. This provides compound no. 6 (315 mg, 97%) with the following characteristics:

### Example 7

### Preparation of heptakis[6-(2-tert-butoxycarbonylaminoethylthio)-6-desoxy-2,3-di-0-hexanoyl]cyclomaltoheptaose (compound no. 7)

This compound complies with Formula IV with m=6, n=1 and in which R represents the tert-butoxycarbonylamino group (NHBoc) and R² represents the hexanoyl group.

This compound is prepared by carrying out the following two steps:

### a) Preparation of heptakis[6-(2-tert-butoxycarbonylaminoethylthio)-6-desoxy]cyclomaltoheptaose

A solution of heptakis(6-desoxy-6-iodo)cyclomaltoheptaose (1 g, 0.53 mmol) or heptakis(6-bromo-6-desoxy)cyclomaltoheptaose (0.84 g, 0.53 mmol) in dried DMF (10 mL), is added to cesium carbonate (1.71 g, 5.25 mmol) and tert-butyl-N-(2-mercaptoethyl)carbamate (5.3 mmol, 1.4 eq). The resulting suspension is heated in Ar at 70° C. for 48 hrs, then cooled to ambient temperature, poured in glacial water and shaken overnight. A solid is obtained i.e. filtered, washed first with distilled water, then ether. The residue is purified by silica gel column chromatography with a mixture CH₂Cl₂-MeOH—H₂O 40:10:1->30:10:1 as eluent. This provides heptakis[6-(2-tert-butoxycarbonylaminoethylthio)-6-desoxy]cyclomaltoheptaose (787 mg, 66%) with the following characteristics:
R_{f}=0.60 (40:10:1 CH₂Cl₂-Me0H-water)
[α]_{D}=+79.7 (c 0.8, MeOH)

### b) Preparation of compound no. 7

A solution of heptakis[6-(2-tert-butoxycarbonylaminoethylthio)-6-desoxy]cyclomaltoheptaose (0.2 g, 89 μmol) in dried pyridine (10 mL), in argon, is added to N,N-dimethylaminopyridine (DMAP, 456 mg, 3.73 mmol, 3 eq). Hexanoyl anhydride (3.4 mL, 14.7 mmol, 4.0 eq) is then added and the reactive mixture is shaken for 5 hrs at 70° C. The reaction is completed by the addition of MeOH (10 mL) and heated at 70° C. for 3 hrs. The mixture is then poured in glacial water (50 mL) and extracted by CH₂Cl₂ (4×50 mL). The organic phase is washed in turn with diluted sulphuric acid (2 N; 2×50 mL) and a saturated solution of NaHCO₃ (4×50 mL), then dried (Na₂SO₄), concentrated and purified by silica gel column chromatography with a mixture of EtOAc-petroleum ether 1:3 as eluent. This provides compound no. 7 (225 mg, 76%) with the following characteristics:
R_{f}=0.45 (1:2 EtOAc-petroleum ether)
[α]_{D}=+84.1 (c 0.9, CHCl₃)

Alternatively, compound no. 7 was prepared from compound no. 1 (164 mg, 50 μmol) by reaction with tert-butyl-N-(2-mercaptoethyl)carbamate (82 μl, 0.5 mmol) and cesium carbonate (163 mg, 0.5 mmol) in dried DMF (3 mL) according to the procedure described above for the preparation of heptakis[6-(2-tert-butoxycarbonylaminoethylthio)-6-desoxy]cyclomaltoheptaose (yield: 88 mg, 49%).

### Example 8

### Preparation of heptakis[6-(2-aminoethylthio)-6-desoxy-2,3-di-O-hexanoyl]cyclomaltoheptaose heptachlorhydrate (compound no. 8)

This compound complies with Formula (IV) with m 6, n=1, R═NH₂ and in which R² represents the hexanoyl group. This compound was isolated in the form of its heptachlorhydrate salt.

Compound no. 6 (71 mg, 0.02 mmol) is treated with a mixture of trifluoroacetic acid (TFA)-water 1:1 (4 mL) at 40° C. for 7 hrs. The resulting solution is concentrated and co-evaporated with distilled water. The residue is picked up by diluted hydrochloric acid (pH 4) and lyophilised. This provides compound no. 8 (60 mg) with the following characteristics:
[α]_{D}=+72.6 (c 1.0, CH₃OH)

### Example 9

### Preparation of heptakis[6-desoxy-2,3-di-0-hexanoyl-6-(2-isothiocyanatoethylthio)]cyclomaltoheptaose (compound no. 9). This compound complies with Formula (VI) with m=6, n=1 and in which R² represents the hexanoyl group.

A heterogenous mixture of compound no. 8 (200 mg, 63 μmol) in acetone (2.4 mL), CaCO₃ (176 mg, 1.76 mmol) and water (3.6 mL), is added to CSCl₂ (69 μL, 0.88 mmol). The suspension is vigorously shaken for 2.5 hrs. Then CH₂Cl₂ (6 mL) and a saturated aqueous solution of NaHCO₃ (6 mL) are added. The organic phase is decanted, dried and concentrated. The residue is purified by silica gel column chromatography with a mixture of EtOAc-petroleum ether 1:4→1:3 as eluent. This provides compound no9 (130 mg, 64%) with the following characteristics:
R_{f}=0.34 (1:3 EtOAc-petroleum ether)
[α]_{D}=+118.8 (c 1.0, CHCl₃)

### Example 10

### Preparation of heptakis[6-(2-tert-butoxycarbonylaminoethylthio)-6-desoxy-2,3-di-O-myristoyl]cyclomaltoheptaose (compound no. 10).

This compound complies with Formula (IV) with m=6, n=1 and in which R represents the tert-butoxycarbonylamino group (NHBoc) and R² represents the tetradecanoyl (myristoyl) group.

A solution of heptakis[6-(2-tert-butoxycarbonylaminoethylthio)-6-desoxy]cyclomaltoheptaose (0.2 g, 89)mol), prepared according to the method described in example 7, in dried DMF (15 mL), in argon at 0° C., is added to N,N-dimethylaminopyridine (DMAP, 456 mg, 3.73 mmol, 3 eq). Tetradecanoyl anhydride (2.18 g, 4.97 mmol, 4.0 eq) is then added and the resulting suspension is shaken for 5 hrs at 70° C. The reaction is completed by the addition of MeOH (10 mL) and again heated at ambient temperature for 48 hrs, then concentrated. The residue is then taken up in a mixture of CH₂Cl₂-MeOH (5:95, 100 mL) and heated to reflux for 1 hr, the solid is decanted and purified by silica gel column chromatography with a mixture of EtOAc-petroleum ether 1:2 as eluent. This provides compound no. 10 (321 mg, 67%) with the following characteristics:
R_{f}=0.56 (1:2 EtOAc-petroleum ether)
[α]_{D}=+49.2 (c 1.0, CH₂Cl₂)

### Example 11

### Preparation of heptakis[6-(2-aminoethylthio)-6-desoxy-2,3-di-O-myristoyl]cyclomaltoheptaose heptachlorhydrate (compound no. 11).

This compound complies with Formula (IV) with m=6, n=1, R═NH₂ and in which R² represents the tetradecanoyl (myristoyl) group. This compound was isolated in the form of its heptachlorhydrate salt.

Compound no. 10 (112 mg, 21.6 mmol) is treated with a mixture of trifluoroacetic acid (TFA)-water 1:1 (4 mL) at 40° C. for 8 hrs. The resulting solution is concentrated and co-evaporated with distilled water. The residue is taken up with diluted HCl (pH 4) and lyophilised. This provides compound no. 11 (102 mg) with the following characteristics:
[α]_{D}=+43.3 (c 1.0, CH₂Cl₂)

### Example 12

### Preparation of heptakis[6-desoxy-2,3-di-0-myristoyl-6-(2-isothiocyanatoethylthio)]cyclomaltoheptaose (compound no. 12).

This compound complies with Formula (VI) with m=6, n=1 and in which R² represents the tetradecanoyl (myristoyl) group.

A heterogenous mixture of compound no. 11 (102 mg, 21.2 μmol) in CH₂Cl₂ (1.6 mL), CaCO₃ (59 mg, 0.59 mmol, 4 eq) and water (6.6 mL), is added to CSCl₂ (23 μL, 0.30 mmol, 2 eq) The suspension is vigorously shaken for 16 hrs. CH₂Cl₂ (15 mL) is then added and the organic phase is decanted, washed with water (6 mL), dried and concentrated. The residue is purified by silica gel column chromatography with a mixture of EtOAc-petroleum ether 1:6 as eluent. This provides compound no. 12 (54 mg, 54%) with the following characteristics:
R_{f}=0.38 (1:4 EtOAc-petroleum ether)
[α]_{D}=+72.3 (c 0.6, CHCl₃)

### Example 13

### Preparation of heptakis[6-desoxy-2,3-di-0-hexanoyl-6-(2-(N′-methylthioureido)ethylthio)]cyclomaltoheptaose (compound no. 13).

This compound complies with Formula (V) with=6, n=1, Z=H, Q=S, T=H, in which W represents the methyl group and R² represents the hexanoyl group.

Et₃N is added to a solution of compound no. 8 (85 mg, 27 μmol) in CH₂Cl₂ (3 mL), until pH 8 is reached. Methyl isothiocyanate (20.5 mg, 0.28 mmol, 1.5 eq) is then added and the reactive mixture is shaken at ambient temperature for 16 hrs. then concentrated. The residue is purified by silica gel column chromatography with a mixture of CH₂Cl₂:MeOH 20:1 as eluent. This provides compound no. 13 (57 mg, 62%) with the following characteristics:
R_{f}=0.41 (20:1 CH₂Cl₂:MeOH)
[α]_{D}=+108.1 (c 1.0, CHCl₃)

### Example 14

### Preparation of heptakis[6-desoxy-2,3-di-0-hexanoyl-6-(2-(N′-(2-hydroxyethyl)thioureido)ethylthio)]cyclomaltoheptaose (compound no. 14).

This compound complies with Formula (V) with m=6, n=1, Z=H, Q=S, T=H, in which W represents the 2-hydroxyethyl group and R² represents the hexanoyl group.

A solution of compound no. 9 (112 mg, 35 μmol) in CH₂Cl₂ (2 mL), is added to a solution of ethanolamine (0.37 mmol, 22.2 μl, 1.5 eq) in CH₂Cl₂ (1 mL), and the mixture is shaken at ambient temperature for 20 hrs. Water (10 mL) is then added and the solution is extracted with CH₂Cl₂ (3×10 mL). All of the organic extracts are dried (Na2SO4), concentrated and purified by silica gel column chromatography with the mixture of CH₂Cl₂:MeOH 9:1->6:1 as eluent. This provides compound no. 14 (82 mg, 64%) with the following characteristics:
R_{f}=0.34 (6:1 CH₂Cl₂-MeOH)
[α]_{D}=+80.9 (c 0.97, MeOH)

### Example 15

### Preparation of heptakis[6-desoxy-6-(2-(N′-(2-N-tert-butoxycarbonylaminoethyl)thioureido)ethylthio)-2,3-di-O-hexanoyl]cyclomaltoheptaose (compound no. 15).

This compound complies with Formula (V) with m=6, n=1, Z=H, Q=S, T=H, in which W represents the 2-N-tert-butoxycarbonylaminoethyl group and R² represents the hexanoyl group.

A solution of compound no. 9 (112 mg, 35 μmol) in CH₂Cl₂ (2 mL), is added to a solution of mono-N-tert-butoxycarbonyl-ethylnediamine (0.37 mmol, 59 μL, 1.5 eq) in CH₂Cl₂ (1 mL), and the mixture is shaken at ambient temperature for 20 hrs. Water (10 mL) is then added and the solution is extracted with CH₂Cl₂ (3×10 mL). All of the organic extracts are dried (Na₂SO₄), concentrated and purified by silica gel column chromatography with a mixture of CH₂Cl₂:MeOH 20:1 as eluent. This provides compound no. 15 (129 mg, 85%) with the following characteristics:
R_{f}=0.27 (20:1 CH₂Cl₂:MeOH)
[α]_{D}=+78.4 (c 1.05, CHCl₃)

### Example 16

### Preparation of heptakis[6-desoxy-6-(2-(N′-(2-aminoethyl)thioureido)ethylthio)-2,3-di-O-hexanoyl]cyclomaltoheptaose heptachlorhydrate (compound no. 16).

This compound complies with Formula (V) with m=6, n=1, Z=H. T=H, in which W represents the 2-aminoethyl group and R² represents the tetradecanoyl (myristoyl) group. This compound was isolated in the form of its heptachlorhydrate salt.

Compound no. 15 (104 mg, 24 μmol) is treated with a mixture of trifluoroacetic acid (TFA)-water 1:1 (2 mL) at 45° C. for 2 hrs. The resulting solution is concentrated and co-evaporatd with distilled water. The residue is taken up by diluted HCl (pH 4) and lyophilised. This provides compound no. 16 (90 mg, 96%) with the following characteristics:
[α]_{D}=+80.7 (c 1.0, DMSO)

### Example 17

### Preparation of heptakis[6-desoxy-2,3-di-0-hexanoyl-6-(2-(N′-(2-α-D-mannopyranosyloxyethyl)thioureido)ethylthio)]cyclomaltohepto se (compound no. 17).

This compound complies with Formula (V) with m=6, n=1, Z=H, Q=S, T=H, in which W represents the 2-a-D-mannopyranosyloxyethyl group and R² represents the hexanoyl group.

A solution of compound no. 8 (85 mg, 27 μmol) in a mixture of water-acetone 2:1 (3 mL) at pH 8 (NaHCO₃) is shaken at ambient temperature for 20 nm. Then a solution of 2-isothiocyanatoethyl-a-D-mannopyranoside (75 mg, 0.28 mmol, 1.5 eq) is added in water (1 mL). After 2 hrs, a precipitate appears i.e. dissolved by the addition of CH₂Cl₂ (0.5 mL) and the reactive mixture is shaken at ambient temperature for 16 hrs. The organic solvents are then evaporated and the aqueous phase is extracted by CH₂Cl₂ (3×5 mL). The organic phase is dried (Na₂SO₄), filtered and concentrated. The residue is purified by silica gel column chromatography with a mixture of CH₂Cl₂:MeOH 1:1->1:2 as eluent. This provides compound no. 17 (49 mg, 38%) with the following characteristics:
[α]_{D}=+75.1 (c 1.0, CHCl₃)

### Example 18

### Preparation of heptakis[6-desoxy-6-(2-(N′-methylthioureido)ethylthio)-2,3-di-O-myristoyl]cyclomaltoheptaose (compound no. 18).

This compound complies with Formula (V) with m=6, n=1, Z=H, Q=S, T=H, in which W represents the methyl group and R² represents the tetradecanoyl (myristoyl) group.

Et₃N is added to a solution of compound no. 11 (65 mg, 14.8 μmol) in CH₂Cl₂ (3 mL), until pH 8 is reached. Methyl isothiocyanate (11.3 mg, 0.16 mmol, 1.5 eq) is then added and the reactive mixture is shaken at ambient temperature for 16 hrs and then concentrated. The residue is purified by silica gel column chromatography with a mixture of CH₂Cl₂:MeOH 30:1 as eluent. This provides compound no. 18 (48 mg, 72%) with the following characteristics:
R_{f}=0.55 (9:1 CH₂Cl₂-MeOH)
[α]_{D}=+60.8 (c 1.0, CHCl₃)

### Example 19

### Preparation of heptakis[6-desoxy-6-[2-[N′-[2-bis[2-(tert-butoxycarbonylamino)ethyl]amino]ethyl]thioureido]ethylthio-2,3-di-O-hexanoyl]cyclomaltoheptaose (compound no. 19)

This compound complies with Formula (V) with m=6, n=1, Z=H, Q=S, T=H, in which W represents the branch element 2-bis[2-(tert-butoxycarbonylamino)ethyl]aminoethyl (see formula below)

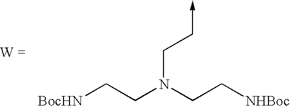

and R² represents the hexanoyl group.

This compound is prepared by carrying out the following two steps:

### a) Preparation of bis[2-(tert-butoxycarbonylamino)ethyl] 2-isothiocyanatoethylamine (see formula below))

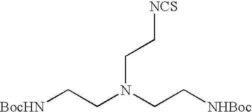

A solution of 2-azidoethyl bis[2-(tert-butoxycarbonylamino)ethyl] amine (0.35 g, 0.94 mmol), prepared as described in document J. Am. Chem. Soc. 2004, 126, 10355-10363, and triphenylphosphine (0.27 g, 1.03 mmol, 1.1 eq) in dry dioxane (10 mL), in N₂, is added to CS2 (0.57 mL, 9.4 mmol, 10 eq). The solution is shaken at ambient temperature for 24 hrs, then concentrated and the residue purified by silica gel column chromatography with a mixture of EtOAc-petroleum ether 30:1 as eluent. This provides bis[2-(tert-butoxycarbonylamino)ethyl]2-isothiocyanatoethylamine (285 mg, 78%) with the following characteristics:
R_{f}=0.72 (1:1 EtOAc-petroleum ether)

### b) Preparation du compound no. 19

Et₃N is added to a solution of compound no. 8 (50 mg, 13.4 μmol) in CH₂Cl₂ (2 mL), until pH 8 is reached. The solution is shaken for 10 mn and then a solution of bis[2-(tert-butoxycarbonylamino)ethyl] 2-isothiocyanatoethylamine (51 mg, 0.13 mmol, 1.2 eq) in CH₂Cl₂ (3 mL) is added. The reactive mixture is shaken at ambient temperature for 24 hrs, then concentrated. The residue is purified by on silica gel column chromatography with the eluent mixture CH₂Cl₂:MeOH 40:1. This provides compound no. 19 (35 mg, 46%) with the following characteristics:
R_{f}=0.37 (20:1 CH₂Cl₂-MeOH)
[α]_{D}=+55.0 (c 1.0, CH₂Cl₂)

### Example 20

### Preparation of heptakis[6-desoxy-6-[2-[N′-[2-bis[2-(tert-butoxycarbonylamino)ethyl]amino]ethyl] thioureidol]ethylthio-2,3-di-O-hexanoyl]cyclomaltoheptaose (compound no. 20).

This compound complies with Formula (V) with m=6, n=1, Z=H, Q=S, T=H, in which W represents the branch element 2-[2-azidoethyl-2′-(tert-butoxycarbonylamino)ethyl]aminoethyl (see the formula below)

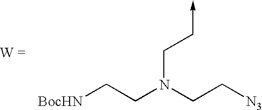

and R² represents the hexanoyl group.

This compound is prepared by carrying out the following steps:

### a) Preparation of 2-(tert-butoxycarbonylamino)ethyl 2-aminoethylamine

A solution of triethylenediamine (10 mL, 93 mmol) in dioxane (40 mL), at 0° C., is added to a solution of Boc20 (2.75 g, 12.6 mmol) in dioxane (40 mL). The mixture is shaken for 4 hrs at 0° C., then allowed to return to ambient temperature. It is concentrated under reduced pressure and the residue is added to (20 mL) and extracted with CH₂Cl₂ (6×40 mL). The organic phase is dried (Na₂SO₄), filtered, concentrated and purified by silica gel column chromatography with a gradient MeOH-> MeOH—NH₄OH (30% in water) 100:3 as eluent. This provides 2-(tert-butoxycarbonylamino)ethyl 2-aminoethylamine (2.25 mg, 88%) with the following characteristics:
R_{f}=0.42 (20:1 MeOH—NH₄OH)

### b) Preparation of 2-(tert-butoxycarbonylamino)ethyl 2-(trifluoroacetamido)ethylamine

A solution of 2-(tert-butoxycarbonylamino)ethyl 2-aminoethylamine (2 g, 10 mmol) in acetonitrile (20 mL) is added to ethyl trifluoroacetate (4.17 mL, 35 mmol). The mixture is shaken at 90° C., at reflux, for 5 hrs, then concentrated and the residue purified by silica gel column chromatography with a mixture of EtOAc-EtOH-water 45:5:3 as eluent. This provides 2-(tert-butoxycarbonylamino)ethyl-2-(trifluoroacetamido)ethylamine (2.58 mg, 64%) with the following characteristics:
R_{f}=0.65 (30:2:1 MeCN—H₂O—NH₄OH)

### c) Preparation of 2-azidoethyl methanesulfonate

A solution of 2-bromoethanol (5 g, 40 mmol) and sodium azide (3.12 g, 48 mmol, 1.2 eq) in water (15 mL) is stirred at 60° C. for 4 hrs, then cooled to ambient temperature and extracted with CH₂Cl₂ (4×20 mL) The organic phase is dried (Na₂SO₄) filtered and concentrated until an oil is obtained i.e. then taken up in CH₂Cl₂ (30 mL) Et₃N (1.5 eq, 8.3 mL) is added and then methanesulfonyl chloride (5.5 g, 48 mmol, 1.2 eq) one drop at a time at 0° C. Allow to return to ambient temperature and shake the mixture for 4 hrs. Add water (40 mL), extract the mixture by CH₂Cl₂ (3×20 mL), then the organic phase is dried (Na₂SO₄), filtered and concentrated. This provides 2-azidoethyl methanesulfonate (5.31 mg, 80%) with the following characteristics:
data by ¹H RMN (300 MHz, CDCl₃): δ 4.33 (t, 2 hrs, ³J_{H,H}=5.0 Hz, CH₂OMs), 3.58 (t, 2 hrs, CH₂N₃) 3.07 (s, 3 hrs, Me)
data by ¹³C RMN (125.7 MHz, CDCl₃): δ 67.7 (CH₂0Ms), 50.2 (CH₂N₃), 38.2 (Me).

### d) Preparation of 2-azidoethyl 2-(tert-butoxycarbonylamino)ethyl 2-(trifluoroacetamido)ethylamine

A solution of 2-(tert-butoxycarbonylamino)ethyl 2-(trifluoroacetamido)ethylamine (1 g, 2.8 mmol) and 2-azidoethyl methanesulfonate (1.02 g, 4.2 mmol) in DMF (10 mL) is added to Cs₂CO₃ (1.37 g, 4.2 mmol) and the resulting suspension is shaken at 50° C. for 24 hrs, then concentrated. The residue is taken up in CH₂Cl₂ (40 mL) and washed with water (20 mL). The organic phase is dried (Na₂SO₄), filtered, concentrated and purified by silica gel column chromatography with a mixture of EtOAc-petroleum ether 1:1 as eluent. This provides 2-azidoethyl-2-(tert-butoxycarbonylamino)ethyl-2-(trifluoroacetamido)ethylamine (531 mg, 80%) with the following characteristics:
R_{f}=0.61 (1:1 EtOAc-petroleum ether)

### e) Preparation of 2-aminoethyl-2-azidoethyl-2-(tert-butoxycarbonylamino)ethylamine

A solution of 2-azidoethyl-2-(tert-butoxycarbonylamino)ethyl-2-(trifluoroacetamido)ethylamine (450 mg, 1.22 mmol) in a mixture of NH₄OH (30%, 4 mL) and MeOH (16 mL) is shaken at 40° C. for 16 hrs, then concentrated. This provides 2-aminoethyl-2-azidoethyl 2-(tert-butoxycarbonylamino)ethylamine, in a quantitative yield, with the following characteristics:
R_{f}=0.59 (10:1:1 CH₃CN-water-NH₄OH)

### f) Preparation of 2-azidoethyl 2-(tert-butoxycarbonylamino) ethyl-2-isothiocyanatoethylamine

A heterogenous mixture containing 2-aminoethyl-2-azidoethyl-2-(tert-butoxycarbonylamino)ethylamine (272 mg, 1 mmol) and CaCO₃ (300 mg, 1.5 eq, 1.5 mmol) in CH₂Cl₂-eau 1:1 (10 mL) is added to C1₂CS (115 1 AL, 1.5 mmol). The mixture is vigorously shaken at ambient temperature for 1.5 hrs, then the two phases are separated, the organic phase is dried (Na₂SO₄), filtered, concentrated and purified by silica gel column chromatography with a mixture of EtOAc-petroleum ether 1:1 as eluent. This provides 2-azidoethyl 2-(tert-butoxycarbonylamino)ethyl-2-isothiocyanatoethylamine (198 mg, 63%) with the following characteristics:
R_{f}=0.72 (1:2 EtOAc-petroleum ether)

### g) Preparation of compound no. 20

Et₃N is added to a solution of compound no. 8 (50 mg, 13.4 μmol) in CH₂Cl₂ (2 mL), until pH 8 is reached. The solution is shaken for 10 mn and then a solution of 2-azidoethyl 2-(tert-butoxycarbonylamino)ethyl-2-isothiocyanatoethylamine (38 mg, 0.13 mmol, 1.2 eq) in CH₂Cl₂ (3 mL) is added. The reactive mixture is shaken at ambient temperature for 24 hrs, then concentrated. The residue is purified by silica gel column chromatography with a mixture of CH₂Cl₂:MeOH 40:1 as eluent. This provides compound no. 20 (55 mg, 80%) with the following characteristics:
R_{f}=0.37 (20:1 CH₂Cl₂-MeOH)
[α]_{D}=+57.4 (c 1.0, CH₂Cl₂)

### Example 21

### Preparation of heptakis[6-desoxy-6-[2-[N′,N′-bis[2-(tert-butoxycarbonylamino)ethyl]thioureido]ethylthio]-2,3-di-O-hexanoyl]cyclomaltoheptaose (compound no. 21).

This compound complies with Formula (V) with m=6, n=1, Z=h, Q=S, in which T and W are identical and represent the 2-(tert-butoxycarbonylamino)ethyl group and R² represents the hexanoyl group.

A solution of compound no. 9 (50 mg, 15.5 mmol) in CH₂Cl₂ (5 mL), is added to Et₃N (15 μL, 1 eq, 0.11 mmol) and bis(2-tert-butoxycarbonylaminoethyl)amine (36 mg, 1.1 eq, 0.12 mmol). The solution is shaken for 6 hrs, then concentrated. The residue is purified by silica gel column chromatography with a mixture of CH₂Cl₂:MeOH 40:1 as eluent. This provides compound no. 21 (76 mg, 92%) with the following characteristics:
R_{f}=0.41 (20:1 CH₂Cl₂-MeOH)
[α]_{D}=+57.0 (c 1.0, CH₂Cl₂)

### Example 22

### Preparation of heptakis[6-desoxy-6-(2-[N′-(2-azidoethyl)-N′-[2-(tert-butoxycarbonylamino)ethyl] thioureidol]ethylthiol-2,3-di-O-hexanoyl]cyclomaltoheptaose (compound no. 22).

This compound complies with Formula (V) with m=6, n=1, Z=H, Q=S, in which T represents the 2-azidoethyl group, W represents the 2-(tert-butoxycarbonylamino)ethyl group and R² represents the hexanoyl group.

This compound is prepared by carrying out the following steps:

### a) Preparation of 2-azidoethyl 2-N-(tert-butoxycarbonyl)aminoethyl)amine

K₂CO₃ (0.40 g, 2.9 mmol) is added to a solution of N-tert-butoxycarbonylethylnediamine (0.47 g, 2.9 mmol) and 2-azidoethyl p-toluenesulfonate (0.70 g, leg, 2.9 mmol) in acetonitrile (10 mL). The mixture is shaken for 12 hrs, then concentrated under reduced pressure and the residue is added to water (40 mL) and extracted with CH₂Cl₂ (2×20 mL). The organic phase is dried (Na₂SO₄), filtered, concentrated and purified by silica gel column chromatography with a mixture of CH₂Cl₂-MeOH 20:1 as eluent. This provides 2-azidoethyl 2-N-(tert-butoxycarbonyl)aminoethyl)amine (0.43 mg, 65%) with the following characteristics:
R_{f}=0.48 (20:1 CH₂Cl₂-Me0H)

### b) Preparation of compound no. 22

Et₃N (15 μL, 1 eg, 0.11 mmol) and 2-azidoethyl-2-N-(tert-butoxycarbonyl)aminoethyl)amine (27 mg, 1.1 eq, 0.12 mmol) is added to a solution of compound no. 9 (50 mg, 15.5 mmol) in CH₂Cl₂ (5 mL). The solution is shaken for 3 hrs, then concentrated. The residue is purified by silica gel column chromatography with a mixture of CH₂Cl₂:MeOH 60:1 as eluent. This provides compound no. 22 (48 mg, 92%) with the following characteristics:
R_{f}=0.24 (60:1 CH₂Cl₂-MeOH)
[α]_{D}=+47.2 (c 1.0, MeOH)

### Example 23

### Preparation of 6^{I}-[2-(tert-butoxycarbonylamino)ethylthio]-6^{I}-desoxy-heptakis(2,3-di-O-hexanoyl)cyclomaltoheptaose (compound no. 23).

This compound complies with Formula (VII) with m=6 and in which R¹ represents the 2-(tert-butoxycarbonylamino) ethylthio group and R² represents the hexanoyl group.

This compound is prepared by carrying out the following four steps:

### a) Preparation of 6^{I}-[2-(tert-butoxycarbonylamino) ethylthio]-6^{I}-desoxycyclomaltoheptaose.

A solution of 6′-deoxy-6^{I}-iodocyclomaltoheptaose (1.42 g, 1.14 mmol) in dried DMF (18 mL), in Ar, is added to cesium carbonate (0.489, 1.48 mmol, 1.3 eq) and tert-butyl N-(2-mercaptoethyl)carbamate (250 μl, 1.48 mol, 1.3 eq). The resulting suspension is heated at 75° C. for 3 hrs, then concentrated. The resulting solid residue is in turn washed with CH₂Cl₂ and acetone and finally purified by silica gel column chromatography with a mixture of CH₃CN-water-NH₄OH 6:3:1 as eluent. This provides 6^{I}-[2-(tert-butoxycarbonylamino)ethylthio]-6^{I}-desoxycyclomaltoheptaose (777 mg, 53%) with the following characteristics:
R_{f}=0.47 (6:3:1 CH₃CN-water-NH₄OH)
[α]_{D}=+115.3 (c 1.0, DMSO)

### b) Preparation of 6^{I}-[2-(tert-butoxycarbonylamino) ethylthio]-6^{II-VII}-hexa-0-tert-butyldimethylsilylcyclomaltoheptaose

A solution of 6^{I}-[2-(tert-butoxycarbonylamino)ethylthio]-6^{I}-desoxycyclomaltoheptaose (378 mg, 0.29 mmol) in pyridine (15 mL) is added to TBDMSC1 (528 mg, 3.5 mmol, 2 eq). The reactive mixture is shaken for 3 days, then poured into glacial water (50 mL) and extracted with CH₂Cl₂ (4×50 mL). The organic phase is in turn washed with diluted sulphuric acid (2 N, 2×50 mL), a saturated aqueous solution of NaHCO₃ (4×50 mL), dried (Na₂SO₄), concentrated and purified by silica gel column chromatography with a mixture of CH₂Cl₂-MeOH-water 50:10:1 as eluent. This provides 6^{I}-[2-(tert-butoxycarbonylamino)ethylthio]-6^{II-VII}-hexa-0-tert-butyldimethylsilylcyclomaltoheptaose (494 mg, 86%) with the following characteristics:
R_{f}=0.38 (45:5:3 AcOEt-EtOH-water)
[α]_{D}=+84.0 (c 1.0, CHCl₃)

### c) Preparation of 6^{I}-[2-(tert-butoxycarbonylamino) ethylthio]6^{II-VII}-hexa-0-tert-butyldimethylsilyl-6^{I}-desoxy-heptakis(2,3-di-O-hexanoyl)cyclomaltoheptaose.

A solution of 6^{I}-[2-(tert-butoxycarbonylamino) ethylthio]-6^{II-VII}-hexa-0-tert-butyldimethylsilylcyclomaltoheptaose (0.85 g, 0.43 mmol) in dried DMF (34 mL), in argon, is added to N,N-AT-dimethylaminopyridine (DMAP, 2.19 g, 17.9 mmol, 3 eq). Hexanol anhydride (5.54 mL, 23.9 mmol, 4.0 eq) is then added and the reactive mixture is shaken for 4 hrs at ambient temperature. The reaction is completed by the addition of MeOH (70 mL) and the shaking goes on for 24 hrs. The mixture is then concentrated, the residue is taken up with CH₂Cl₂ (150 mL), washed in turn with diluted sulphuric acid (2 N; 2×50 mL) and a saturated solution of NaHCO₃ (4×50 mL), then dried (Na₂SO₄), concentrated and purified by silica gel column chromatography with a mixture of EtOAc-petroleum ether 1:20->1:9 as eluent. This provides 6/-[2-(tert-butoxycarbonylamino)ethylthio] 6^{II-VII}-hexa-0-tert butyldimethylsilyl-6^{I}-desoxy-heptakis(2,3-di-O-hexanoyl)cyclomaltoheptaose(806 mg, 56%) with the following characteristics:
R_{f}=0.62 (1:8 EtOAc-petroleum ether)
[α]_{D}=+72.1 (c 1.0, CHCl₃)

### c) Preparation of compound no. 23

A solution of 6^{I}-[2-(tert-butoxycarbonylamino)ethylthio]-6^{II-VII}-hexa-0-tert-butyldimethylsilyl-6^{I}-desoxy-heptakis(2,3-di-O-hexanoyl)cyclomaltoheptaose (78.2 mg, 23.3/μmol) in dry tetrahydrofurane (3 mL) is added to tetrabutylammonium fluoride (TBAF, solution 1 N in THF, 167 μl, 167 μmol, 1.2 eq). The reactive mixture is shaken at ambient temperature for 16 hrs, then concentrated and the residue purified by silica gel column chromatography with a gradient EtOAc -> EtOAc-EtOH mixture as eluent. This provides compound no. 23 (38 mg, 61%) with the following characteristics:
R_{f}=0.47 (20:1 EtOAc-EtOH)
[α]_{D}=+94.4 (c 1.0, CHCl₃)

### Example 24

### Preparation of 6^{I}-(2-aminoethylthio)-6^{I}-desoxy-heptakis(2,3-di-O-hexanoyl)cyclomaltoheptaose chlorhydrate (compound no. 24).

This compound complies with Formula (VII) with m=6 and in which R¹ represents the 2-aminoethylthio group and R² represents the hexanoyl group. This compound is isolated in the form of its heptachlorhydrate salt.

Compound no. 23 (71 mg, 0.02 mmol) is treated with a mixture of trifluoroacetic acid (TFA)-CH₂Cl₂ 1:1 (0.26 mL) at ambient temperature for 3 hrs. The resulting solution is concentrated and the residue is purified by silica gel column chromatography with a gradient EtOAc-EtOH 20:1-> EtOAc-EtOH-water 45:5:3 as eluent. The purified product is taken up with diluted HCl (pH 4) and lyophilised. This provides compound no. 24 (36 mg, 64%) with the following characteristics:
R_{f}=0.19 (20:1 AcOEt-EtOH)
[α]_{D}=+80.4 (c 1.0, CH₂Cl₂)

### Example 25

### Preparation of 6^{I}-desoxy-6^{I}-[2-[N′-(2-a-D-mannopyranosyloxyethyl)thioureido]ethylthio]-heptakis(2,3-di-O-hexanoyl)cyclomaltoheptaose (compound no. 25).

This compound complies with Formula (VII) with m=6 and in which R¹ represents the 2-[N′-(2-α-D-mannopyranosyloxyethyl)thioureido]ethylthio group (see formula)

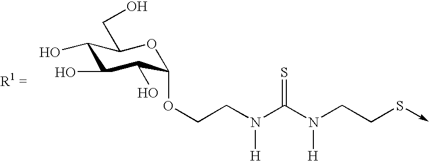

and R² represents the hexanoyl group.

A solution of compound no. 24 (113 mg, 42 μmol) in a mixture of water-acetone 2:1 (4 mL) at pH 8 (NaHCO₂) is shaken for 20 nm at ambient temperature. Then a solution of 2-isothiocyanatoethyl-a-D-mannopyranoside (22 mg, 83 μmol, 2 eq) in water (0.5 mL) is added. After 2 hrs, a precipitate appears which is dissolved by the addition of CH₂Cl₂ (0.5 mL) and the reactive mixture is shaken at ambient temperature for 48 hrs. The organic solvents are evaporated and the aqueous phase is extracted with CH₂Cl₂ (3×5 mL). The organic phase is dried (Na₂SO₄), filtered and concentrated. The residue is purified by silica gel column chromatography with a gradient EtOAc-EtOH 20:1-> EtOAc-EtOH-H₂O 45:5:3 as eluent. This provides compound no. 25 (46 mg, 39%) with the following characteristics:
R_{f}=0.52_(45:5:3 EtOAc-EtOH-H₂O)
[α]_{D}=+92.5 (c 1.0, CH₂Cl₂)

### Example 26

### Preparation of 6^{I}-[2-(N′-(2-(cyclomaltoheptaose-6^{I}-desoxy-6^{I}-yl)ethylthio)thioureido) ethylthiol-6^{I}-desoxy-heptakis-(2,3-di-O-hexanoyl)cyclomaltoheptaose (compound no. 26).

This compound complies with Formula (VII) with m=6, R² represents the hexanoyl group (C₅H₁₁CO₂) and R¹ the following group:

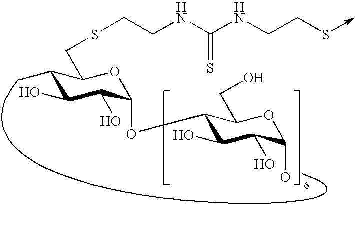

It is prepared by the following series of steps starting with compound 24 (Example 24) and the 6^{I}-[2-(tert-butoxycarbonylamino)ethylthio]-6^{I}-desoxycyclomaltoheptaose prepared in example 23a.

### a)6^{I}-(2-Aminoethylthio)-6^{I}-desoxy-cyclomaltoheptaose trifluoroacetate.

A solution of 6^{I}-[2-(tert-butoxycarbonylamino)ethylthio]-6^{I}-desoxycyclomaltoheptaose (300 mg, 0.23 mmol) in a mixture of TFA-water (1:1, 2.7 mL) is shaken at ambient temperature for 2 hrs, then concentrated. This provides 6^{I}-(2-aminoethylthio)-62-desoxy-cyclomaltoheptaose trifluoroacetate i.e. directly used in the following step.

### b)6^{I}-Desoxy-6^{I}-(2-isothiocyanatoethylthio) cyclomaltoheptaose.

A solution of 6^{I}-(2-aminoethylthio)-6^{I}-desoxy-cyclomaltoheptaose trifluoroacetate (270 mg, 0.20 mmol) in a mixture of acetonitrile-water (1:1, 6 mL), at 0° C., is added to calcium carbonate (82 mg, 0.82 mmol) and thiophosgene (31 μL, 0.41 mmol). The reaction is shaken for 2 hrs, then filtered, the solvents are evaporated and the residue is purified by silica gel column chromatography with a mixture of CH₃CN-water 9:1->3:1 as eluent. This provides 6^{I}-desoxy-6^{I}-(2-isothiocyanatoethylthio) cyclomaltoheptaose (129 mg, 50%) with the following characteristics:
R_{f}=0.36 (3:1 CH₃CN-water)
[α]_{D}=+56.1 (c 1.0, DMSO)

### c) 6^{I}-Desoxy-heptakis(2,3-di-O-hexanoyl)-6^{I}-(2-isocyanatoethylthio)cyclomaltoheptaose.

A solution of compound 24 (215 mg, 82.6 μmol) in a mixture of dichloromethane-water (1:1, 2 mL), at 0° C., is added to calcium carbonate (25 mg, 0,25 mmol) and thiophosgene (9.5 μL, 0.12 mmol). The reaction is shaken at ambient temperature for 4 hrs. Dichloromethane (5 mL) is then added. The organic phase is separated, washed with water (3 mL), dried (Na₂SO₄) and concentrated. The residue is purified by silica gel column chromatography with a mixture of CH₂Cl₂-MeOH 15:1->9:1 as eluent. This provides 6^{I}-desoxy-heptakis(2,3-di-O-hexanoyl)-6^{I}-(2-isocyanatoethylthio) cyclomaltoheptaose (130 mg, 60%) with the following characteristics:
R_{f}=0.28 (9:1 CH₂Cl₂-MeOH)
[α]_{D}=+89.6 (c 0.3, CHCl₃)

### d) Compound no. 26.

Sodium bicarbonate (solution saturated in water) is added to a solution of compound 24 (121 mg, 46.5 mmol) in DMF (2 mL) until pH 8 is reached, and the reactive mixture is shaken at ambient temperature for 20 nm. A solution of 6^{I}-desoxy-6^{I}-(2-isothiocyanatoethylthio)cyclomaltoheptaose (86 mg, 70 μmol) in DMF (0.5 mL) is added and the resulting solution is shaken at ambient temperature for 24 hrs. The solvent is evaporated at reduced pressure, the residue is taken up in dichloromethane (5 mL), washed with water (2 mL), the organic phase is dried (Na₂SO₄) and concentrated. The residue is purified by silica gel column chromatography with a mixture of CH₂Cl₂-MeOH 10:1 as eluent, then by filtration chromatography on gel (Sephadex LH-20, MeOH). This provides compound 26 (80 mg, 45%) with the following characteristics:
[α]_{D}=+91.6 (C 1.0, CHCl₃)

Alternatively, compound 26 (105 mg, 60%) was prepared by reaction between 6^{I}-(2-aminoethylthio)-6^{I}-desoxy-cyclomaltoheptaose trifluoroacetate (66 mg, 51 μmol) and 6′-desoxy-heptakis(2,3-di-O-hexanoyl)-6^{I}-(2-isocyanatoethylthio)cyclomaltoheptaose (121 mg, 46.4 μmol) in DMF (3 mL) at pH 8 (NaHCO₃) following the same procedure.

### Example 27

### Formulation of White Homogenous Nanospheres

The method used to prepare white homogenous nanospheres consists in introducing an organic phase of acetone (or tetrahydrofurane) and an amphiphilic cyclodextrin derivative from the invention (1 mg·mL⁻¹) in an aqueous phase of equivalent volume or a double volume (distilled water), under magnetic stirring (500 r.p.m.) at 25° C. After nanoprecipitation, the organic solvent is eliminated under reduced pressure at +35° C. The aqueous suspension presenting an opalescent to milky appearance (according to the products used) is then concentrated until the final volume desired is obtained, then filtered on a 0.8 μm filter (Millex AA, Millipore, France).

The nanoparticule suspensions were tripled.

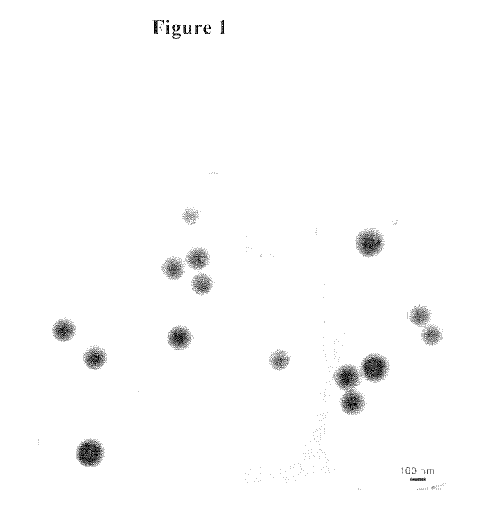

FIG. 1 represents a cryo-transmission photograph of nanospheres observed by electronic transmission microscopy after cryofracture.

The colloid suspensions are then characterised as regards the granulometry (mean size and polydisperion) and, when they incorporate loaded amphiphilic cyclodextrins, by their zeta potential. The characterisation is carried out using the following methods:

Measurement of the size of nanoparticles.

The quasi-elastic diffusion of light is used. A Zetasizer 3000 apparatus equipped with a K7132 correlator (Malvern Instruments, RU) was used. The conditions for measurement are as follows: angle of detection 90°, temperature 25±0.1° C., apparent viscosity 0.89 mPa·s. Before measuring the size, samples are diluted in distilled water if necessary. The hydrodynamic diameter (mean Z) and polydispersion index (PI) of the nanoparticles in suspension are calculated in intensity by the cumulant method by carrying out 3 measurements on the same sample. The value of the hydrodynamic diameter is calculated using the measurement of the translational coefficient of diffusion of particles with Brownian movement. An NanoZS apparatus (Malvern Instruments) was also used to measure the size of the nanocapsules. In the same way, the samples are diluted before measurement, if necessary in water added to Montanox 80 (trade mark).

### b) Measurement of the Zeta Potential of the Nanoparticles

A zetasizer 3000 apparatus (HeNe 10 mW laser at 632.8 nm) (Malvern Instruments, Malvern, RU) was used. The measurements were taken at a temperature of 25±0.1° C. after dilution of the samples in a solution of sodium chloride (10³M). The zeta potential values are obtained by carrying out 3 measurements on the same sample.

The studies on the physical stability of particles were carried out over periods ranging from 1 month to 8 months. The corresponding data for a series of amphiphilic cyclodextrins in the invention are indicated in Table 1 providing the granulometric characteristics (mean size and polydispersion index or mean PI), with evolution over time, of different homogenous nanospheres derived from the compounds indicated in the left column, the reference number for said compounds corresponding to those of the compounds described in the above examples.

| | initial | | 3 months | | 6 months | | 8 months | |
|---|---|---|---|---|---|---|---|---|
| | Mean size ± | | Mean size ± | | Mean size ± | | Mean size ± | |
| | standard | | standard | | standard | | standard | |
| | deviation | | deviation | | deviation | | deviation | |
| compound | (n = 3) | | (n = 3) | | (n = 3) | | (n = 3) | |
| no. | (nm) | PI | (nm) | IP | (nm) | IP | (nm) | PI |
| 1 | 251 ± 62 | 0.08 | | | | | 236 ± 13 | 0.03 |
| 2 | 238 ± 29 | 0.02 | | | | | 182 ± 22 | 0.01 |
| 3 | 181 ± 5 | 0.08 | 157 ± 8 | 0.06 | 206 ± 17 | 0.18 | | |
| 4 | 217 ± 17 | 0.04 | 206 ± 20 | 0.03 | 227 ± 26 | 0.06 | | |
| 5 | 244 ± 13 | 0.13 | 232 ± 22 | 0.05 | 242 ± 27 | 0.03 | | |
| 6 | 171 ± 9 | 0.03 | 157 ± 8 | 0.06 | 198 ± 12 | 0.04 | | |
| 7 | 283 ± 29 | 0.06 | | | | | 194 ± 2 | 0.02 |
| 9 | 123 ± 2 | 0.03 | 138 ± 2 | 0.09 | | | | |
| 10 | 212 ± 14 | 0.11 | 207 ± 1 | 0.07 | 269 ± 64 | 0.32 | | |
| 12 | 72 ± 32 | 0.31 | | | | | | |
| 13 | 115 ± 31 | 0.16 | | | | | | |
| 26 | 100 ± 20 | 0.06 | | | | | | |

### Example 28

### Formulation of Mixed White Nanospheres

The method used to prepare mixed white nanospheres consists in introducing an organic phase of two amphiphilic cyclodextrin derivatives in acetone, in variable proportions (1 mg·mL⁻¹), in an aqueous phase of equal volume (distilled water), under magnetic stirring (500 r.p.m.) at 25° C. After nanoprecipitation, the organic solvent is eliminated under reduced pressure at 35° C. The aqueous suspension presenting an opalescent appearance is then concentrated to the final volume desired and then filtered (0.8 pm, Millex AA, Millipore, France). The nanoparticle suspensions are tripled. The colloid suspensions are then characterised in terms of mean size and zeta potential, following the procedures described above and then stored at +6° C.

In a typical example, the organic phase consists of a solution of compounds no. 2 and no. 8 (1 mg·mL-1), in relative proportions of 4:1 and 7:3, in acetone with distilled water (water-acetone proportion 1:1 v/v) as aqueous phase. In the case of a 4:1 proportion of compounds no. 2 and no. 8, in an independent series of experiments, white nanospheres are obtained with a mean size ranging from 110 to 138 nm with PI that vary from 0.16 to 0.27 and a zeta potential that varies from 43 to 47 mV. In the case of a 7:3 proportion of compounds no. 2 and no. 8, in an independent series of experiments, white nanospheres are obtained with a mean size that ranges from 88 to 162 nm with PI that vary from 0.14 to 0.2 and a zeta potential that varies from 40 to 45 mV.

In another typical example, the organic phase consists of a solution of compounds no. 25 and 26 (1 mg·mL⁻¹), in relative proportions of 1:4, in acetone with distilled water as aqueous phase (proportion water/acetone 1:1 v/v). In the case of a 1:4 proportion of compounds no. 25 and 26, in a series of independent experiments, white nanospheres are obtained with a mean size that ranges from 95 to 120 nm with PI of 0.03 to 0.08.

### Example 29

### Formulation of White Nanocapsules

The method used to prepare white nanocapsules first consists of the preparation of an acetone phase containing a small fraction of capric/caprylic triglycerides (Miglyol 812 (trade mark)), an amphiphilic cyclodextrin derivative from the invention, a lipophilic non ionic surfactant (Montane 80 (trade mark)) and a hydrophilic phase containing distilled water and a hydrophilic non ionic surfactant. The organic phase is then introduced in the hydrophilic phase under magnetic stirring (500 r.p.m.) at 25° C. After nanoprecipitation, the organic solvent is eliminated under reduced pressure at 35° C., The aqueous suspension presenting a milky appearance is then concentrated until the final volume desired is obtained. The batches of colloid suspension were tripled. The nanoparticles were then characterised in terms of mean size and then were stored at +6° C.

In a typical example, acetone, Miglyol 812 (trade mark) (proportion acetone-Miglyol 812 (trade mark) 100:1 v/v), Montane 80 (trade mark) (4 mg·mL⁻¹) and compound no. 3 (2 mg·mL⁻¹) is used as organic phase, and distilled water (proportion water-acetone 2:1 v/v) containing Montanox 80 (trade mark) (2 mg·mL⁻¹) is used as aqueous phase. In this case, in a series of independent experiments, white nanoparticles are obtained with a mean size ranging from 203 to 255 nm with PI that vary from 0.04 to 0.24.

### Example 30

### Formulation of Nanospheres Loaded with Active Ingredient (Diazepam)

The method used to prepare loaded nanospheres of host molecule, here an active ingredient (diazepam), consists in introducing an organic phase containing acetone, an amphiphilic cyclodextrin derivative from the invention and diazepam (DZ) in an aqueous phase containing distilled water with or without non ionic hydrophilic surfactant (for example poloxamer 188), under magnetic stirring (500 r.p.m.) at 25° C. After nanoprecipitation, the organic solvent is eliminated under reduced pressure at 35° C. The aqueous suspension presenting an opalescent appearance is then concentrated to the final volume desired.

The batches of colloid suspensions were tripled. The nanoparticles were then characterised according to granulometry. The quantity of diazepam bound to the nanospheres was assessed by the difference between the quantity of diazepam present in the final colloid suspension and the quantity of diazepam present in the supernatant. The diazepam was assayed by spectrophotometry at 285 nm. The level of encapsulation or association of active ingredient with the nanoparticles (TEₑₓₚ) and the yield or efficacy of encapsulation (RE) were thus assessed. The aqueous suspensions were stored at +6° C.

In a typical example, the organic phase consists of acetone, compound no. 3 (1 mg·mL⁻¹) and diazepam (0.5 mg·mL⁻¹), and the aqueous phase consists of distilled water (water-acetone proportion 1:1 v/v). In this case, in a series of independent experiments, loaded nanospheres are obtained with a mean size ranging from 135 to 175 nm with PI that vary from 0.01 to 0.02, TEₑₓₚ vales ranging from 24 to 28% and RE values between 74 and 89%.

TEₜₕ corresponds to the mass percentage of DZ initially introduced (DZᵢ). It is determined as follows:

100×DZᵢ(mg)/(DZᵢ+CD)mg

TEₑₓₚ corresponds to the percentage of DZ really associated with the nanoparticles (DZₐ)

RE: 100×DZₐ(mg)/DZᵢ(mg)

### Example 31

### Formulation of Nanocapsules Loaded with Active Ingredient (Diazepam)

The method used to prepare loaded nanocapsules of host molecule, here an active ingredient (diazepam), first consists in preparing an acetone phase containing a small fraction of triglycerides with capric/caprylic chains (Miglyol 812 (trade mark)), an amphiphilic cyclodextrin derivative from the invention, a non ionic lipophilic surfactant (Montane 80 (trade mark)), diazepam (DZ) and a hydrophilic phase containing distilled water and a non ionic hydrophilic surfactant. The organic phase is then introduced in the hydrophilic phase under magnetic stirring (500 r.p.m.) at 25° C. After nanoprecipitation, the organic solvent is eliminated under reduced pressure at +35° C. The aqueous suspension presenting a milky appearance is then concentrated to the final volume desired. The batches of colloid suspensions were then tripled. The nanoparticles were then characterised in terms of mean size and stored at +6° C.

In a typical example, acetone, Miglyol 812 (trade mark) (acetone—Miglyol 812 (trade mark) proportion 100:1 v/v), Montane 80 (trade mark) (4 mg·mL⁻¹) and compound no. 3 (2 mg·mL⁻¹), and diazepam (1 mg·mL⁻¹) were used as organic phase and distilled water (water-acetone proportion 2:1 v/v) containing Montanox 80 (trade mark) (4 mg·mL⁻¹) as aqueous phase. In this case, in a series of independent experiments, loaded nanocapsules were obtained with a mean size ranging from 215 to 225 nm with PI varying from 0.03 to 0.07, RE values between 85 and 93%.

### Example 32

### Preparation of Complexes with Compounds No. 8 or 16 and the Plasmid pTG11236, Characterisation and Transfection Ability

### a) Preparation

The plasmid pTG11236 (pCMV-SV40-luciferase-SV40 pA), used in the preparation of DNA complexes and for transfection tests is a plasmid with 5739 pairs of bases. The quantities of compounds used are calculated to obtain a DNA concentration of 0.1 mg·mL⁻¹ (303 μM in phosphate equivalent) and the desired Nitrogen/Phosphate (N/P) ratios. Tests for values of N/P=5, 10, 30 and 50 were carried out both for compound no. 8 and for compound no. 16. As a reference transfection formulation (positive control), we used polyplexes formulated with the same plasmid and the polycationic polymer polyethyleneimine (branched PEI, 25 kDa), and naked DNA as a negative control.

For the preparation of the complexes, the DNA is diluted in HEPES (20 mM, pH 7.4) until a final concentration of 303 μM in phosphate equivalent, then the quantity of compound no. 8 or no. 16 necessary, obtained from a stock solution of 20 mg·mL⁻¹ in DMSO-water 1:2 (v/v) is added in order to obtain the desired N/P ratio. For the PEI, a 0.1M stock solution in water is used. The mixtures are vortexed for 10 sec and used in the characterisation and for the transfection tests, as described below.

### b) Characterisation of DNA-Compound No. 8 or No. 16 Complexes

b-1) Measurement of Size And Polydispersity of Nanoparticles of DNA.

The almost-elastic diffusion of light is used as described above by employing a CoulterN4 MD apparatus. Measurements are directly taken on the above preparations or at a DNA concentration of 0.1 mg·mL⁻¹ (303 μM in phosphate equivalent).

In the case of DNA nanoparticles formulated for an N/P ratio of 10 and 30 with compounds no. 8 or no. 16, in a series of independent experiments, nanoparticles are obtained with a mean size ranging from 150 (Standard Deviation —SD—=100) to 200 (SD=190) nm. By way of comparison, the polyplexes formulated with PEI for an N/P ratio of 10 have a mean size of 160 nm (SD=100).

b-2) Electrophoretic Analysis of Nanoparticles of DNA.

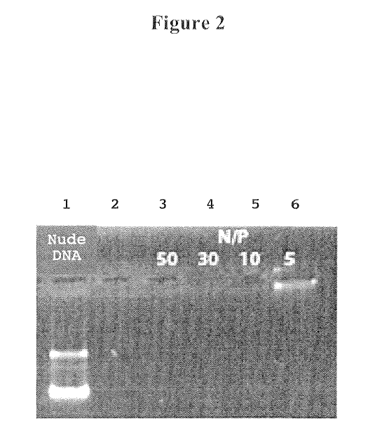

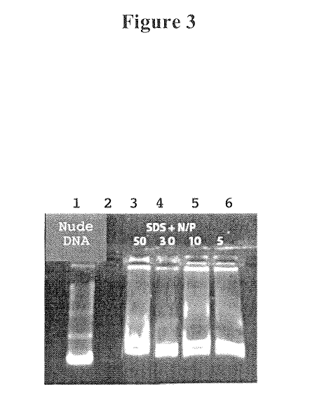

10 μl of each formulation of complexes prepared as mentioned above are diluted once in the loaded buffer (glycerol+bromophenol blue+Tris-acetate-EDTA or TAE). 1 μl of this solution are deposited on a gel with 8% agarose. The rest of the sample is diluted once with a decomplexing solution with 8% sodium dodecyl sulfate (SDS) in TAE, then 10 μl are also deposited on a gel. The electrophoresis is carried out for 90 nm at 50V. The gel is then developed with ethidium bromide (Sigma) in TAE (201 of a solution of ethidium bromide with a concentration of 10 mg·mL⁻¹ in 200 mL of TAE). The DNA is also visualised and photographed with an UV transilluminator. FIGS. 2 and 3 are photos of electrophoretic analyses, carried out on agarose gel in the presence of ethidium bromide, of DNA complexes/nanoparticles obtained with compound no. 8 according to an N/P ratio of 5, 10, 30 and 50 respectively.

Analysis of FIG. 2 shows the total absence of bands corresponding to the DNA as regards the nanoparticles of DNA derived from the preparation with an N/P ratio≧10 (row 3 to 5) compared with naked DNA i.e. clearly visible (row 1). This result indicates the total complexation of the DNA. However, for an N/P ratio=5 (row 6), the DNA still remains partially accessible at the intercalation by the ethidium bromide.

Very similar results were obtained with complexes formulated with compound no. 16, forming complexes with the DNA as of an N/P ratio of 5, since the DNA is no longer accessible at the intercalation of the ethidium bromide.

The integrity of the plasmid in each sample is confirmed by electrophoresis on gel after decomplexation with SDS, as shown in FIG. 3 for a time of incubation of 10 nm.

### Example 33

### Tests of In Vitro Transfection of Nanoparticles of DNA

Murine embryo cells BNL-CL2 are grown in 96-well plates until a density of 2×10⁴ cells/well in a DMEM culture medium (Dulbelcco's Modified Eagle Medium, Gibco-BRL) containing 10% foetal calf serum (SVF; Sigma) at 37° C. in a humid atmosphere with a proportion of 5% CO₂/95% air.

The complexes between compound no. 8, no. 16 or polyethyleneimine (PEI), and plasmid pTG11236 are diluted in 100 μl of DMEM so as to obtain 0.5 μg of DNA in the well.

The culture medium is removed and replaced by 100 μl of solution of nanoparticle complex/DNA in DMEM. After 4 hrs and 24 hrs, 50 and 100 μL of DMEM containing 30% and 10% of FCS, respectively, are added. After 48 hrs, the transfection is stopped, the culture medium is removed and the cells are washed with PBS (2×100 μL), then lysed with 50 μL of lysis buffer (Promega, Charbonnières, France). The lysates are frozen at −32° C. until the luciferase and protein assay.

The luciferase assay is used to assess the efficacy of transfection. This assay is based on a chimioluminescence reaction whose principle is based on oxydation by luciferase of its substrate (luciferine) with concomitant production of a photon. The activity of the luciferase is measured with a Biolumat LB96P WMP100 apparatus (BERTHOLD) for 10 sec after the injection of 50 μl of reagent containing luciferine (Prmèga assay kit) in each well containing 10 μl of cell extract. For each assay, a standard series from 1 ng/μl to 1 ng/μl is carried out with R-luciferase (Bio-Rad), allowing the RLU measurements to be converted into femtogrammes (fg) of luciferase per well. The assay of the proteins in each well (see below) is used to convert these measurements into fg of luciferase per mg of protein.

The protein assay is based on the BCA test (Pierce). The protein assay is carried out on 15 μl of cell lysate to which are added 300 μl of Pierce reagent (BCA Protein Assay Reagent). It consists of a colorimetric assay which is carried out with UV/visible MRX spectrophotometry (DYNEX Technologies). For each assay, a standard series is carried out with BSA (Bovine Serum Albumin, BIO-RAD), of 0 to 30 μg per well. The reading is taken at 570 nm, after 30 nm of incubation at 37° C. and 15 nm at ambient temperature. With this standard BSA series, the OD may be expressed in mg of protein/well.

The ratio of the quantities of proteins corresponding to the transfected and non transfected (100% cell viability) cells is used to assess the cell viability of the complexes (expressed in %).

All of the formulations are tested at least 3 times. The results obtained were processed using STATGRAPHICS Plus5.1 software (trade mark). Anova analysis of variance is carried out on the values of the efficacies of transfection (LoglO(fg luciferase/mg protein)) and on the cell viability. It is possible to analyse the impact (on the transfection or cell viability) of the variation of one or several factors by applying a mono- or multifactorial analysis of variance, respectively. The method used to compare the means is the HSD (honestly significant difference) intervals by Tukey. This HSD technique by Tukey is used to compare all of the pairs of means and demonstrate differences with an alpha risk.

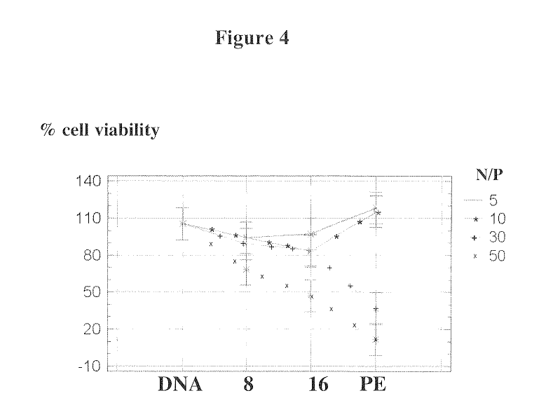

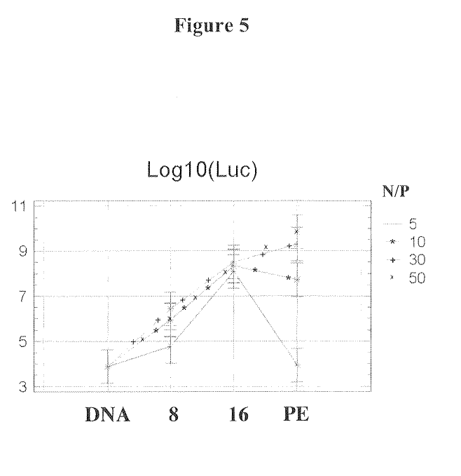

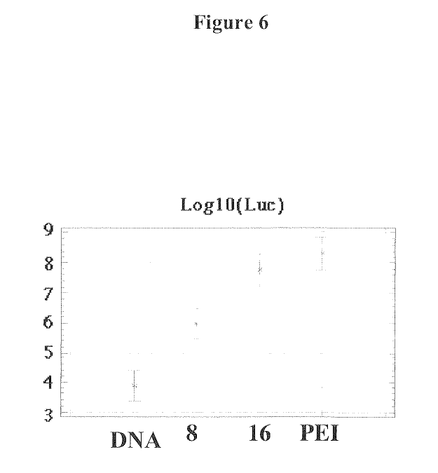

Two factors, the type of complexation agent (compounds no. 8, no. 16, or PEI) and the N/P ratio, were analysed as a source of variation of percentages of cell viability (FIG. 4) and the logarithm of the levels of transfection (FIGS. 5 and 6).

FIG. 4 represents the means and 95% significance intervals by Tukey and cell viability after transfection of BNL-CL2 cells by complexes formulated with compounds no. 8, no. 16 or PEI and plasma DNA pTG11236, as a function of the N/P ratio ranging from 5 to 50, and determined for a plasmid concentration of 0.5 μg/well.

Analysis of FIG. 4 indicates excellent cell viabilities after transfection of complexes formulated with compound no. 8 or no. 16.

For N/P ratios>10, the transfections obtained with the complexes formulated with DNA and compounds no. 8 or no. 16 reveal that the cell viability is significantly higher than that obtained after the transfection of cells, with the same N/P ratios, by the polyplexes derived from PEI/DNA complexes.

FIG. 5 represents the means and 95% significant intervals by Tukey of the logarithmic transformation of the expression of luciferase (fg of luciferase/mg of protein) by BNL-CL2 cells transfected with complexes formed by compounds no. 8, no. 16 or PEI and plasma DNA pTG11236 for a N/P ratio=5, 10, 30 and 50, and for 0.5 μg of DNA per well on BNL-CL2 cells.

FIG. 6 represents the means and 95% significance intervals by Tukey of the logarithmic transformation of the expression of luciferase (fg of luciferase/mg of protein) by the BNL-CL2 cells transfected with complexes formulated with compounds no. 8, no. 16 or PEI and the plasma DNA pTG11236 for N/P=10 and 0.5 μg of DNA per well.

Analysis of FIGS. 5 and 6 indicates levels of transfection that are, for N/P ratios>10, about 100 times higher than those with naked DNA, and about 100 times lower than those measured for polyplexes with N/P 10 polyplexes formulated with PEI.

For complexes formulated with compound no. 16, this statistical analysis indicates levels of transfection that are about 10,000 times higher than those with naked DNA, and equal to those measured for N/P 10 polyplexes formulated with PEI that correspond to the optimum in the reference formulation (FIG. 6).

1. A cyclodextrin having the following Formula (I): 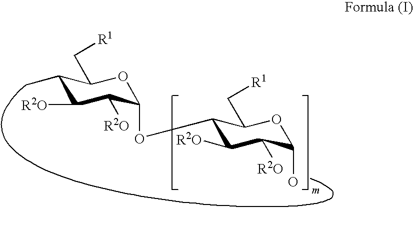
in which:
m=5, 6 or 7
the identical or different R¹ radicals, represent:
(1) an OA group in which A represents a hydrogen atom, an alkyl radical in C_{I} or C₂ to C_{I2}, aryl in C₆ to C₂₀, or even a protector group, such as a silyl group, in particular tert-butyldimethylsilyl or tert-butyldiphenylsilyl, in particular the OA group represents a hydroxyl group (OH);
(2) a functional group chosen among:
a halogen atom;
an azide group (N₃);
a sulphide group of the SR³ type, in which R³ is:
(i) an alkyl substituent in C_{I} or C₂ to C_{I2} or aryl in C₆ to C₂₀ or;
(ii) a element of biorecognition such as an amino acid derivative, a peptide, a monosaccharide, an oligosaccharide, an element of multiplication with several branches, which branches may carry glucidic groups that may be identical or different, a visualisation probe or elements of fluorescent or radioactive detection, or other functional groups;
(iii) CH₂—(CH₂)ₙ—B with n=1 to 5, B is:
NHX and X is a hydrogen atom, an alkyl group in C_{I} or C₂ to C_{I2} or aryl in C₆ to C₂₀, or
NZC(=Q)NTW, where Z represents a hydrogen atom, an alkyl group in C_{I} or C₂ to C_{I2} or aryl in C₆ to C₂₀, Q represents an oxygen atom or a sulphur atom and T and W, identical or different, represents a hydrogen atom, an alkyl substituent in C_{I} or C₂ to C_{I2}, aryl in C₆ to C₂₀ or an element of cell recognition such as an amino acid, a peptide, a monosaccharide, an oligosaccharide or even an element of multiplication with several branches carrying glucidic groups that may be identical or different, or charged groups such as ammonium groups;
an amine group of the NHR⁴ type, in which R⁴ is:
(i) a hydrogen atom;
(ii) an alkyl substituent in C_{I} or C₂ to C_{I2} or aryl in C₆ to C₂₀;
(iii) an R^{a}—C(=0)- substituent, in which R^{a} represents an alkyl radical in C_{I} or C₂ to C₂₁, or aryl in C₆ to C₂₀ or;
(iv) a carbamate, urea or thiourea substituent, possibly substituted by at least one group chosen from along the alkyl groups in C_{I} or C₂ to C_{I2} or aryl in C₆ to C₂₀, elements of biorecognition, visualisation or detection, in particular such as those mentioned above for R³
the identical or different R² radicals represent: an alkyl group in C_{I} or C₂ to C_{I2}, aryl in C₆ to C₂₀, or R^{a}—C(=0)- in which R^{a} represents an alkyl radical in C_{I} or C₂ to C₂₁, or aryl in C₆ to C₂₀;
with at least one of the R¹ radicals different from OH,
as well as their salts and their isomers;
where each of the aforementioned alkyl radicals may be linear, branched or cyclic, saturated or unsaturated;
and each of the aforementioned aryl radicals is possibly substituted.

2. A cyclodextrin according to claim 1, in which R², R³ and/or R⁴ are chosen among the benzyl, phenyl, allyl, methyl, ethyl, propyl, butyl, pentyl, hexyl groups or superior homologues comprising up to 12 linear, branched or cyclic, saturated or unsaturated carbon atoms, groups that may comprise other neutral or charged functional groups.

3. A cyclodextrin according to claim 2 in which R² and/or R⁴ are chosen among the acetyl, propionyl, butyroyl, pentanoyl, hexanoyl groups or homologues comprising up to 22 linear, branched or cyclic, saturated or unsaturated carbon atoms, these groups may bear other neutral or charged functional groups.

4. A cyclodextrin according to any one of claims 1 to 3, in which all of the R¹ radicals are identical and represent halogen atoms, in particular chosen among iodine or bromine.

5. A cyclodextrin according to any one of claims 1 to 3, complying with the following Formula (III): 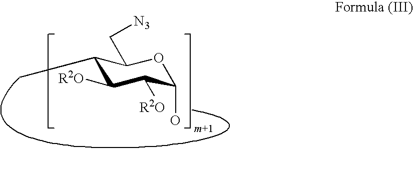
in which m and R² are as defined in claims 1 to 3.

6. A cyclodextrin according to any one of claims 1 to 3, complying with the following Formula (IV): 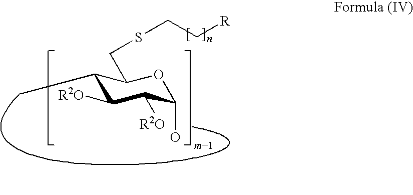
in which m and R² are as defined in claims 1 to 3,
n=1, 2, 3, 4 or 5, and R represents an anime function such as:
(i) an NHY amine group, Y representing a hydrogen atom or even, a carbamate substituent, an alkyl substituent in C₁ or C₂ to C₁₂, or a R^{a}—C(=0)- substituent in which R^{a} represents an alkyl radical in C₁ or C₂ to C₁₂ or aryl in C₆ to C₂₀, or;
(ii) a quaternary —NY₃ ammonium group, Y representing an alkyl substituent in C₁ or C₂ to C₁₂;
where each of the aforementioned alkyl radicals may be linear, branched or cyclic, saturated or unsaturated;
and each of the aforementioned aryl radicals is possibly substituted.

7. A cyclodextrin according to claim 6, in which n=2 and R represent the tert-butoxycarbonylamino (NHBoc) group or NH₂.

8. A cyclodextrin according to any one of claims 1 to 3, in which the R¹ radicals are identical complying with the following Formula (V): 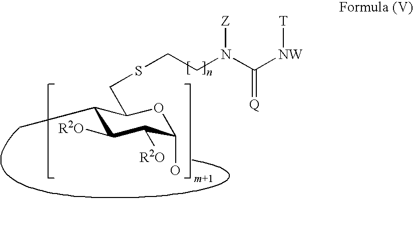
in which m and R² are as defined in claims 1 to 3
n represents a whole number chosen among 1, 2, 3, 4 or 5,
Z represents a hydrogen atom, an alkyl group in C₁ or C₂ to C₁₂ or aryl in C₆ to C₂₀,
Q represents an oxygen atom or a sulphur atom and
T and W, identical or different, represent a hydrogen atom, an alkyl group in C₁ or C₂ to C₁₂, aryl in C₆ to C₂₀ or an element of cell recognition such as an amino acid, a peptide, a monosaccharide, an oligosaccharide or even an element of multiplication with several branches bearing glucidic groups that may be identical or different, or charged groups such as ammonium groups;
where each of the aforementioned alkyl radicals may be linear, branched or cyclic, saturated or non saturated;
and each of the aforementioned aryl radicals is possibly substituted.

9. A cyclodextrin according to claim 8, in which n=2, Q represents a sulphur atom, Z and T represent a hydrogen atom and W represents the methyl group.

10. A cyclodextrin according to claim 8, in which m=6, n=2, Q represents a sulphur atom, Z and T represent a hydrogen atom and W represents a group chosen among 2-hydroxyethyl, 2-(tert-butoxycarbonylamino)ethyl, 2-aminoethyl, possibly protonated, 2-(α-D-mannopyranosyloxy)ethyl, 2-[2-azidoethyl-2′-(tert-butoxycarbonylamino)ethyl]aminoethyl, 2,2-bis[2-(tert-butoxycarbonylamino)ethyl]aminoethyl.

11. A cyclodextrin according to claim 8, in which m=6, n=2, Q represents a sulphur atom, Z represents a hydrogen atom, T and W are identical and represent the 2-(tert-butoxycarbonylamino)ethyl group.

12. A cyclodextrin according to claim 8, in which m=6, n=2, Q=S, Z=H, T represents the 2-azidoethyl group and W represents the 2-(tert-butoxycarbonylamino)ethyl group.

13. A cyclodextrin according to claim 8, in which all of the R² radicals represent a hexanoyl group.

14. A cyclodextrin according to claim 1 in which m=6 and all of the R² radicals represent the hexanoyl group and/or the tetradecanoyl (myristoyl) group.

15. A cyclodextrin according to any of claims 1 to 3 complying with Formula (VI): 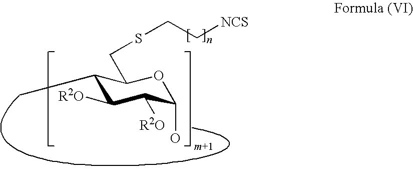
in which m and R² are as defined according to any of claims 1 to 3, and n represents an integer chosen among 1, 2, 3, 4 or 5, and in particular in which n=2, m=6 and R² represent the hexanoyl group or tetradecanoyl (myristoyl) group.

16. A cyclodextrin according to claim 1 to 3, complying with the following Formula (VII):
in which m, R¹ and R² are as defined according to any of claims 1 to 3.

17. A cyclodextrin according to claim 16, in which the R¹ radical represents the 2-(tert-butoxycarbonylamino)ethylthio, 2-aminoethylthio group or 2-[N′-(2-α-p-mannopyranosyloxyethyl)thioureidol]ethylthio.

18. A cyclodextrin according to claim 16, in which m=6 and all of the R² radicals represent the hexanoyl group.

19. A cyclodextrin according to claim 18 comprising at least one host molecule, in particular forming an inclusion complex.

20. A method for the preparation of cyclodextrin as defined according to claim 1, comprising steps consisting in:
(i) introducing at least one group on at least one of the carbons bearing the primary hydroxyl or protecting at least one of the primary hydroxyls of the starting compound, in particular a cyclodextrin;
(ii) introducing at least one R² group on at least one secondary hydroxyl carried by carbon in position 3 of the monomers forming a cyclodextrin;
(iii) recovering at least one cyclodextrin as defined according to any of claims 1 to 19.

21. A method according to claim 20, to prepare a cyclodextrin that complies with Formula (I) in which all R¹ groups represent a halogen atom, in Formula (III) or Formula (IV) in which R represents NHY, Y represents a carbamate group or R^{a}—C(=0)- in which R^{a} represents an alkyl radical in C₁ or C₂ to C₁₂ or aryl in C₆ to C₂₀ and the R² radical represents an R^{a}—C(=0)- group in which R^{a} represents an alkyl radical in C₁ or C₂ to C₂₁, or aryl in C₆ to C₂₀,
in which a selectively halogenated, azidated or functionalised cyclodextrin derivative with NHY groups in primary alcohol position with an acid anhydride, in particular in N,N-dimethylformamide, in the presence of a base, preferably N,N-dimethylaminopyridine;
where each of the aforementioned alkyl radicals may be linear, branched or cyclic, saturated or non saturated;
and each of the aforementioned aryl radicals is possibly substituted.

22. A process according to claim 20, to prepare a cyclodextrin that complies with Formula (III) as defined in claim 5, in which a halogenated cyclodextrin derivative as defined in claim 2 is made to react with an azide anion.

23. A process according to claim 20, to prepare a cyclodextrin as defined in claim 6, in which a halogenated cyclodextrin derivative as defined in claim 2 is made to react with cysteamine, a ω-aminothiol, or one of their derivatives, in the presence of a base, such as triethylamine or cesium carbonate.

24. A process according to claim 20, to prepare a cyclodextrin as defined in claim 6 in which R represents a primary amine group (NH₂) in which the carbamate group is hydrolysed in a percursor as defined in claim 7 in which R represents an NHBoc group.

25. A process according to claim 20, to prepare a cyclodextrin that complies with Formula (V) as defined in claim 8 in which a precursor of Formula (IV) as defined in claim 6 in which R represents NHY, where Y represents a hydrogen atom or an alkyl substituent in C₁ or C₂ to C₁₂ or aryl in C₆ to C₂₀, is made to react with an isocyanate or an isothiocyanate of general formula W—NCQ, Q represents an oxygen atom or a sulphur atom and W has the meaning indicated in claim 8;
where each of the aforementioned alkyl radicals may be linear, branched or cyclic, saturated or non saturated;
and each of the aforementioned aryl radicals is possibly substituted.

26. A process according to claim 20, to prepare a cyclodextrin that complies with Formula (IV) in which R represents a primary amine group (NH₂) consisting in hydrolysing the carbamate group in a precursor of Formula (IV) in which R represents an NHBoc group.

27. A process according to claim 20, to prepare a cyclodextrin of Formula (V) in which Q=S and Z=H, as defined in claim 7, in which a precursor as defined in claim 15 is made to react with an amine of general formula WNHT, W and T having the meaning indicated in claim 8.

28. A process according to claim 20, to prepare a cyclodextrin that complies with Formula (VII) in which one of the R¹ radicals is different from the hydroxyl and the others represent OH, consisting in:
(i) selectively introducing a functional group on one of the primary positions of the cyclodextrin;
(ii) protecting the other primary hydroxyls with a protector group, in particular in silylether form;
(iii) then introducing the substituents on the primary hydroxyls; and
(iv) possibly hydrolysing the protector groups.

29. A nanostructure comprising at least one of the cyclodextrins according to claim 1.

30. A nanostructure according to claim 29, incorporating, comprising, being associated or forming a complex, with at least one host molecule.

31. A nanostructure according to claim 29 or 30 coming in the form of nanospheres, nanocapsules or nanoparticles.

32. Nanostructures, in particular nanospheres and/or nanocapsules according to claim 31, also comprising at least one host molecule, in particular at least one pharmacologically active molecule.

33. A nanocapsule according to claim 31, enclosing or containing an organic phase, in particular such as Miglyol 812 (trade mark).

34. Nanoparticles according to claim 31 also comprising at least one nucleic acid, in particular selected from the group containing DNA (linear or plasma), RNA (and in particular interfering RNA -RNAi- or even (<silencing >> -RNAsi-, micro-RNA), modified nucleic acids, such as the ribonucleotides or desoxyribonucleotides presenting a sugar group or a modified carbon group, or even synthetic analogs of nucleotides.

35. A method for the preparation of nanostructures, and in particular nanospheres, comprising steps consisting of:
(i) the addition of at least one water-miscible organic solvent containing at least one compound of Formula (I) of identical or different chemical formula, with an aqueous solution, the volume of water varying in particular from one to two times the volume of organic solvent, under stirring;
(ii) then after nanoprecipitation, i.e. the formation of nanostructres, the elimination of the organic solvent.

36. Method according to claim 35, in which, in step (i), at least two organic solutions are added, in variable proportion, containing cyclodextrins of Formula (I) of different chemical formulae respectively.

37. A method for the preparation of nanostructures, in particular nanocapsules, comprising the following steps:
(i) the preparation of an acetone phase containing a small fraction of triglycerides, preferably an acetone: triglyceride proportion ranging from 1,000:1 to 10:1, a preparation of amphiphilic cyclodextrin of Formula (I) of identical chemical formula, at least one non ionic lipophilic surfactant, and a hydrophilic phase containing distilled water and at least one non ionic hydrophilic surfactant;
(ii) the introduction of the organic phase in the hydrophilic phase under magnetic stirring;
(iii) then after nanoprecipitation, i.e. the formation of nanocapsules, the elimination of the organic solvent.

38. A method according to claim 37, in which in step (i), at least two cyclodextrin preparations are added, in variable proportion, containing cyclodextrins of Formula (I) of different chemical formulae respectively.

39. A method for the preparation of nanostructures, in particular nanospheres and/or nanocapsules, comprising at least one host molecule, in which the nanostructures are as defined in claim 32, comprising the following steps:
(i) the introduction of at least one organic phase containing a water-miscible solvent, such as acetone, a cyclodextrin derivative, or, alternatively, several preparations comprising cyclodextrins complying with Formula (I) of different chemical formulae in variable proportions, and the active ingredient, in an aqueous phase, possibly with a surfactant, in particular a non ionic hydrophilic surfactant, while shaking,
(ii) then after nanoprecipitation, i.e. the formation of nanocapsules or nanospheres, the elimination of the organic solvent.

40. A cosmetic, food and/or pharmaceutical, composition comprising at least one cyclodextrin according to claim 1 and/or one nanostructure according to claim 29 and an active compound.

41. A pharmaceutical composition according to claim 40, containing per unit dose from 50 mg to 500 mg of cyclodextrin according to claim 1 and/or nanostructures according to claim 29 and a pharmacologically active host molecule in a molar proportion of cyclodextrin derivative/host molecule that may range from 50:1 to 1:500, in particular from 25:1 to 1:10, in particular from 20:1 to 1:1, or even 10:1 to 1, 5:1.

42. (canceled)

43. (canceled)

44. An in vitro method for the transfer of host molecules in cells, in particular eukaryote cells consisting in:
(i) putting in contact the nanostructure/host molecule complex and/or cyclodextrin/other host molecule complex with cells;
(ii) leaving in contact the cells with the nanostructure/host molecule complex and/or cyclodextrin/host molecule complex for a time T preferably between 4 and 72 hours;
(iii) removing the culture medium and washing the cells.
